SEQUENCE LISTING (35 sequences)

SEQ ID NO: 1 - protein, 1000 aa, Artificial Sequence
  Randomly generated sequence, created by ExPASy WWW server tool
SQSPKPSSQSQSQPPSSKKSKQQQQPKSPSSSPQSQSPSSKPSSSSPQQPSKSSKSPKPPSPSPPPSKKPKSPSKPSPKPPSPPKSKSPKQPQSSSQSQSSSSKSSQPPSPPSSQKPSQSQSSSQPKPSSPKPQSSPQKQSPSQPKKSQKPKKQKKPQQPSSPQPKPQSQPQPPQSSSSKSSPQSSQQSSQSPPPPPPSSSSPPKSKPSKPQSQKPPSPSSKPKSKSSPQKSSSPSPKSKSPQPPKQQSPPKPPPKSPQPKPSPPSSPKKPKPPPSPKSQSSSQPSPKSKSQPPSSSQPSPSSSQQSQSPQPSSQKPPQSPSQKSKKSSPPSPPPPPSPPSQKQPPPPSSPKPPPQQSPQKSPKSPKQSKQSPPSQPSPPPPPSSPQPKPSSQPKPQSKQPQQPSKSKPPPPQSKPPPQSPSKPQQQPSPPKPPSKPKPPPQPKSKSKKPKQSPKSPKSPPKKSSQKSSSPPQSPKKQKSQSPSSSQPPKPPKPPSSPPPPSSSKPPSKKPQSSSSSPSPSQQPQPSSPSQPPPSSPPPPQPSQPPSPSSKKKQKQPQQKPPQQQSQKSKQQKQQKSSPPPSSSSPSKKPPPPSSPKSQKKKPPSQPSPQPSSSQSPSQQSQSKPSSSPQPSPQPKSQSPQSQKPSPQSSPSKSKPPSSSSQPKPSSPSQQPSQPPKSSKSKQPPPPSQQPSPKQSSSSPKKKPPQPPKKQSQQKPPPQPPPPSPPPPQQKSSSSKSKQKSKPSPSQSSPSPPSPPPPQSPKQKSSKSPPKQPSPPQPQSPKKQPQKSPPSQSPSSQSSPQPSPPPSSSQSPPPPKSSQSSSSSSKPPPSPKPPPQPSPQSSQPQKKSQPSSSKSPKPPPPSSKPPKQSSPKPSQPPSSQSKQQKQSKKKSKKKPSPPKKSKQPQPQSPSKSPKKPSSKSSKSPPKSSPSSPSKSPPQKPPSQKSSKPPPPSSSQSKPQQSPKPSKPSPPSSSSPPQQQSSSSKQSQSPPPPSSPSPSPS

SEQ ID NO: 2 - protein, 1000 aa, Artificial Sequence
  Randomly generated sequence, created by ExPASy WWW server tool
KPPPKSQKKSSKKPQQKSSKSPKSKKSSKPQKQKSKPPKSKSQPPKKSKQPSKKKKPSKKPPKSKQQKPKKKSPSPPPQSPSSKKKPSSSPKPKKKPSPPSSKSKKPKSPSPSKSKQQSPQKSPSPKSKQQSSKKSPSSSQSPPKSKKSSKKSSKKSPSQKKQPQPQSSPPKPPQPKPSPKPSSSPPPKPQQPPKPPSQKSPPKPKPSSPSQKKSSQKSKQKQPPPPSSKPSKSKPKKKKSSPKQPPPSPQQSSKPKKSSSSQKSPPQKQQKPSSQSSSPPPQSKSKKSSPKKSPPKSKPSQPQPSSSKPPKSKSSQQSSSSQKKPSQQQPSSPKKPQSPPSPPPKPPPPQSSSSKSPPKKSKSSPKQPPSPPSQSSQQSSKSSPSPPKKKKQPKQSKPKQQPSKQSKKKPPPQPKKSPQKQKSQPKKQQQKPSPQPKSSSKSSKPSSPKKKPQSSPPQQKQPSKPPQSPSPQKSQKSPQPPSPPKSPQPPKKSKSSSSKSKKSSSQKPPPQPKPSQPKSPPSQSKKPSKPPSPPSKPKQPQSPKSKQQSSPPSSPSKSKQKPPKQSSQPSQPPPKSPSPSSPKSKPKPKPSQSSKSSKKKPSKPPSQSPSQKKSSKSPPPKSKPPPSQSPKSKKKSPSQKSKKKKQKKPKPKPPPSQKKQQKSSSPPPSKKSSPSKSKPPSPPSKKSSKSPPPKKKPPPQSPSPKQSPQPKKPSKSSPPQQSPKKKSPKQPPSKPKPKPPPKQKPSSKPQKSSSKSKKPKPPSKQSQKKSKQPQSPQPSSKQKPKPKQSSPPKSKSKKKPPQKKPSQPKSSKPSSKPKKKQPPPPQPKPPQKKSKQSSKSPPPPSKKSKPSKKSQQQKSQSPSPKSSPPSPKPKKSPPPSSSPSSSPSSPKPPSSQSQKKQSPKQQPSKQKSSPPKKSKKPKKPPPSPSSKKKKPKKSKSKKPPSPKQKKSKQKSKPKPPKQPQSSQPPKQPKPQQQSQSSQPPQQSQKPQKPKSPQQS

SEQ ID NO: 3 - protein, 1000 aa, Artificial Sequence
  Randomly generated sequence, created by ExPASy WWW server tool
QSSSPPKSSSQSKSSSSSSSSPSPKSPSSPSKPPPPSKKKPKSKKKQSSPKSSKPKKPKQKKSPPPQKPKKSPSKPKSKPSSSKKKKSQQQSSQKSQSKQPKKPQPSPKKPKSPKKPPKPQPKSSPKQSKQKPSKKKPSSKPKSKSKKKSQKPKQSKKSSSKPPSKSKKKQPKPKKKSKSSSSKSSKSPSKSKSPQSSKSSPPKKPKPKKPKPKSSKSPKSPPKKKPQSQKQPKSQSPQPQKKPKQSSKQKPKSKKSPKKPPKKSKPKSPPPPKKPKPKKSSKQPKSQSSQKKPKPPPPSPPKQKPQKSSSPPKQQSKKPSPPQKPKPKSSPSPSKSSQSKKKKPKKPKQSPPQKPPSKQSPQKPKSSSPPKKKKSSKKQKKKQKKQKSSQSKPSQKPPSKPKSSSSKKKQSKKKKPPQKSSKKQQSPPKQSPKPSPKKKKPKKKQKKSPKQSQPKKPKPSKPQKSQKKSPSPKPPPQPKPQKKSPPKPKPKSPSPPPSQKPKKPSKPQQSPQKKPPPKSQKKPKPPKKKSKSSSPPQSKQQKKKKKKSPKSKKSKQPQPKQKKKSKPKSPSQKPKQSSSKQKKSPKPKPSPKSSKPQPKKKKKPSKKKKKKKQKPPPQSKKPKSPPPKPKPKSSSKKPPPKPSKPQSKKQSKSKKKPPKQKKKPKKSPKKKKKPPSSKSSPKSPPSQQSPPPPKQSKQPPSQSKKPPKPPKKKSSKKKKKSKKPQKQPKKKSSSKQSKSKPPSPSQPPSPSKPPSPKKKSPSQSKPKQKSPSKSSKSKQSKPSKQQPKQKPQSSQKPKSPKSKKKSQKKQSSSPPKSKSQQPKPSQKKPPKQQSSKSPQKSSKQKPSKPSSPKPQSKQSKQQKKKKQSKQPPKQKKPSKSKKPPPKPPPKSKPKQKKPQKKPKSSKKPQQPSPSSPSSKSSKKSKSKQKPPPQPPPSQKKKKPPPKSQKKPKKKKSSPSKKKPPKKKSPSQSSQKSKSSSQSPPQQPPQKPKKSKQKKK

SEQ ID NO: 4 - protein, 1000 aa, Artificial Sequence
  Randomly generated sequence, created by ExPASy WWW server tool
SSKPKKSPPSKKQSQSKKSKPKKKKSQKPKKSSPKKKSKSSKKPSPPQPSKQPKQQSPSKQSKSPKSQKPPSPPKKKQKKPSKQPKSPKPPKSKSQQPKPKPQQPKKKPKPSKPPPPSSQKQQKSKSPSQKKKKPSKKPKKKQPKQSPSSKPSSQPKQPPQKKKKPKPKKKKKQKQPKKPKKKKSPKKKPKPPKSKKKKPKSSKKSKPQKPSPPKSPKPKPKPKKKPKSKKSKSSKPKPPSKKKPPPSPPSSPKQKSKSPPKKKPKQKPKQKSKSSSPQPKPPSSPKKKKKQSKSKKPSKKSPPKKKKSQQKSSKKPKKPKKSKKSSKKKSKPQSKPKSSKKKKSSSKSSPKKPKPQQPKKKKQQKKKKSSKPKQKKSQKKPSKKKPKKPKQKKSKKSPPKKQSKQPPQKKSKKKQKPPSQKKSQSSPKPKPPQKPKKKSPKPPKKPQKKPKSKQSSSKPSKPPPPKKPPKKPKPKKKKKKSKKSSKKKKQPSPKKPKSKKKKKSSKPSKPSQQKSPKSKPSSSPQSKQPKQSSSSSKKPKKPPSKSKQPSSKSPKSPPPKPSQKPPPQKKPKQKKSKKPPKKKKKPQKPKKSSPSPPPSPKQKKKQPPSKQPKSKKSSQKKSSKSKKKKKKKPPKKSKSPPSQSKSKPSPPPKKPKKQSSQQSKSQQSSKPKPKPKKPPPKQSPSPSSQKKKKPKSKKPSSPSSPKSSSPSSSPSKSSKQKPSSPSKPKKPKKKPKKKPKKPKKQPKQKPKKPPPSKKPKPPSKSQSKKPKQKKSSPKKKKSKKSKKSKQQKQQKKKSQKKSKSSPPKSKKQKQSKKPKQPKKKQSKSPKKQKKPKSSPSQKQQQKKKKQPSKSSKKPKQKKKSKQSKPKQPKKSSPPKSPSKQSKKSPSKSQKPQSKKSPKSKKKSSKKKKKKKKPKKPKKKPKKSKSSSQKKSKQPKSPSQKSSKKKKPKQSSKKKQKKQKQKKKQPSSKPQPKKKQPKKKQKKPKKKKSPKSPKPK

SEQ ID NO: 5 - protein, 1000 aa, Artificial Sequence
  Randomly generated sequence, created by ExPASy WWW server tool
KKKQPKKSQQKKKKKKQSKPKQKKPPSSKPPKQKKKQPKKSPSKSSSKKKQKSPKPQKKPKKPKKPKKSKKQPQQPPSKPSPQSKSKQPQQKKPPKPKPPKKPKKKKQPSQKQSKPPKSQSQKKSSKQKSPSKPKQKSSKKKKKKPSSSPSKSKKKKPKSKPPKKSKPKKKKKSQSKKPKKKKPKQQQKPKPSKQQKPKPSSKKSSPKKKPKQKPKPQPKPKKPKPPKPKQKKKSKPKPKSPKKKQQQQPKPPQKSPKKSPPKKPKPKKSSPSKSPSKPKKQKPKKPSSQKKPKSKSPPKKQSKKSKSKSKKKSPSSKKSKPKKSSPKKPKSKKQSKSKSQKPKSKQSSPKQKKKSQKSKPQKSKKKSSPKKQKSKKKKSPKKPSKPPKKKPPKSKQSKKKQSPKPKPPSPSPKPKKKSKKKKKKQPSSKKQPKKPSKKKKQSPSKQPKSKSSKKKPPKKQPKKPKKKKQSSKKPKKSPQKKSKKPQSSPKKSPSKQPKKKKPKKPKKPKKKKPQSSPSKPPPKSQSKQKSPPKSSSKKKQKKPKPKKKKKPSKKKPPPSKKPKKSKKSKSKKKSKKKSPPKKSKKKQPKPPKKSKKKSSKQSKPKKSPKPKSKKKSKKQKSSSKKSPPPKSKPPKPSQPPKSKKKKPPSKKKPKKQKSSQKPKSSQKKKPPKPKKQPKSKKPKKPKKQQQKKPPKKKKKKKKKKPKPKKPPKPQSKSKKKKKSPPSPPSPKKKKKQKKKSKKKKPKKKPQKKSSKQKKKKPSSSKPKSQSKKSSKKPKQSKQKKSQSKKSSSKSKPQKKSKKKKKKKPKKKKKKKSKSKSSQSQKKKKKSPKKKKKKSKKKKSKKPPKPKKQSKKSKSKPPPSKPKSSKSKPKKPPKKKKQKKKQKSKPSKKSPSKPPSKPSKQKKKSQKKQPQPPKKQPPKSKPKPPKPQKSSKKKKKPSKKPPKKKSKKQKKKKSQSQKKSSSQKPKSSKSSQKKPKKKSKSSKQKSKKQKSKKKPK

SEQ ID NO: 6 - protein, 1000 aa, Artificial Sequence
  Randomly generated sequence, created by ExPASy WWW server tool
EEPSPSPPESSSEPPPPPPPQPPEPPQQSEQPQESSPSQSQSEPSEQQQESSSSEQESSSPPESQEEPQSEQPSSPPEPQPQSQSSQPPPSESPSQQSEPPPEQSQSPSSPSSSSQQSQPPSSEPSEPSPSSPQSSPSPSPQQSPEESESQPQSPSSQSPPQPPSEPSPPQSSEPPEPPSSEPQPSPSSPPQPESPSSSSSPPSPPSPQEPSPEQPPPPPPPQSPESPPSEPPQSPPEQEPEQPPEPESSPPQSQSSEPQSQPEPQSSEQSEESESQQEPPSSPEPPSPEEEQPSPSSPSPPQSPPEPPPSSEPESSPSSESPSEQSPPEPSEQSSQSPSPSPPQQEQSPPSQSSPEPPSSPEPEESPPPEPESSSSPSSSQPEEQPSSPSPPSPPSSSQSSPSSQSPSSPEESPSPPPPPPESEPSPQQPSPPQQEPPPSQSSPSQQSPPPPSSPPPSEQPPQEPQPPSQSSQPPEPSSQSEPSPPPQSPPQPESPQPSSSSQPSSEPPSPSSSPPEPSPSPEQPPPSPSQEEPSQEPSQSESSEQSQSPPSPSESSQSPPQSSSSPQSPEPQPPPSESQESQPPPSESQPSPEESSPSSQSEQPSQSQEPQQSPPQPSPEQPESEQESPSPSEESESSSSQSPPPSPQEPSPPSESQSSPSSPPQPSSSQESPSSQPQPQSQSPPQQPQQSPPPSPPPQQSEEQEQESEPQEPQPQSSPESPSSESESESSPEQPPQPPPSPEPPPPSPSPSPPSESQPSQPQPSSSSESPEESPQPPPEESPSSSSSEEPPQPEEEQSSEPSSQSPSSSPSPSQSESQSQSSSESSSSESESQSPEPEEPEPPSQESPPEQPQQEQQPEESSSSSSSPQSEPPEEPSPQQQQSSSSSPESSPPPEQEQPEQSPQPPSQSPQSSSQESSEPQPEQQSPEEEPSPSQSSSSSPSPPPPEQSEQPEPPESPEPQQQSPQPPSSQEPEEPEPQSPPESEPPEEESQSPQPQ

SEQ ID NO: 7 - protein, 1000 aa, Artificial Sequence
  Randomly generated sequence, created by ExPASy WWW server tool
EQPEPPSESPSPSPPSSESSPPPSSEPSSPQSQSPEEEPSQSQPSESSPEPSPEQSSPSEEEQPPESSQSQESQEPPESPPQQPSPPSQESSEQESPEQEESEPPSEEPEPPSESSEEEQEQSPQSPSSEPEPEQSQESPSSSESPSPEESPPQPPEPPESPPPSPEQEQQPEEESPPQPESSPSESSSPESPQEPPSSPPPESSEEEESQESSPQQSEEQSSSPSPSQSESQQESPEPPSQPPSSSEPSSPSPSPEPEPQQPQQQSQPESPSPSPQQPSQPSEESPESPEPPSSEPSEPSEEPESEQEPSSPPESSEPEQSQEEPEPEQSQSESSPEESPESSEQQQEPEPPSPSSQSPPSSPPSSEPPSPPEPSPSSESPEQQQEEQPSEEPQSSSEEQSQSSEPPEPSPQSSPSPQSEPPEQEQEEPEQSEPQPEPPEQSPEPSSSPEQQPEPPPQSSSPPSQEESSPPEESSPEESSEEPSSEQQQEPSSPQEPEPSSQPPEPPQQPEPEPSEPPPSQSEPPPSPPEEQQSSPPEPEPPPESPSQEEPPSSSQEEQQEPESQEPEESQPEPPSPPQPEEESPQSEEPPSPSQPSPSEEQSEPSQQQEPSQPSESPESPQESEQEPEEPESSPEEESPSPQSPPSSPPPESEEQPEEQPPQQSPEPPPSSPESPESEPEESPPEESEEQPQQPSQEEPPESQESSSPQSSSEESPPPQESEQPEPESEQEPPPEQQPEQSEQSSEQQPPPESSQPPSSSSESEEEEESSEQEPSSSEEPESSESSSEQSSESEESEEEPPQQQEESPPSEEEEQQQPPPEPESESPEQSQPSEPSPSSESQEEPQEPSSSPSPEEPQEESEESPPESPESSQPSPSSQEPPESEESQPEQESSPEEPEPPPPEPEEPPPPPSPEPEEEEQPQPSQQSSSQEEESESSEEPSSEPSSEPEESSSSSPSSEQQSESQEEPEEESEEPPPSSESPEEEEEPSEPPESSEP

SEQ ID NO: 8 - protein, 1000 aa, Artificial Sequence
  Randomly generated sequence, created by ExPASy WWW server tool
SPEQPEPQPEPEQESEPEPSEPPPSQEEESEEEEQSEQPEEESSEPSPESSPSPQEPSPQQEPPSEPQQESEPSQSPSSESEQSEEQEPQEESESEESPESSPSSEPSEEESEQSESSEEEEPPSPPSPEEESPESQEQQEPEQQSEPEEESSSSPSPEPSEEPPPESEPSEESPPSEQSEPEPPPESSEPPQQEQESEESSSPPESEPPEQSSEPEEEQQSEEEESPEEESSEESSPEQSSSSSEEESSEEPESPEEEEPSQPEQPQQSPPQESPPEESQEPPSESSSSEQSSESQSQSPSSSSEPQEPQPPEPSSQEEPEPPEQEPEPSQPSEESSPSSEPEESPPEEESESSESEESEEEEEEEESPSPSPQEPSSQPPSEEPSEEPSPEEQESEEEESPSSSEQEEPSQSEQQSPPSSPPESEQSQEEEPEEEEQPPEPSQSPEESESEEQQSSESEPPQSPPEEPEPEQQQSSSEESEQESEPSQEESESESEESEESSPSSSPQPEEPESEEEQPSPSPESQEPEESEPSEEPSQSPEEEEEEPEPEPQQSEEEQPQESSQQEEEEPPESEQQPSSEQEESEEPQQEEPSESQPQPPESSPPSPPPPEEPSQEESEQEPEEEQSPPEPEEQEPSPSESEESPPESESSEEQQEESEPESEEEPPQQSEEQQSQPEEEEEEQSEEPSSSPPEPPQQEPSSPSEQPPQPEEPEPEEESEEPSPEQPSESSEPPESPEEPSPPPPSSEESESESEQPEEQPESEEPPSSPSESSEEPEEEPEEEQPSEPQPPSEQPSPPEEPQEESEEEPPSEEPSQSESPEPEPSPSSPPPQEPEQPSSSEQSPPEPSEQSPPSQEEPEEEPSQSEQESEEQPQEEPPQPSPEPSPQEPSEPEPEEPPEEEPPQPPPSSEPEEQESSSPEPQQPQPSSSPEEEPPEESPEPSPQPEPESEPEEEQSPSEQEPEEEESQEPSSPQEPEEEQSESESPSPEPEPEPEE

SEQ ID NO: 9 - protein, 1000 aa, Artificial Sequence
  Randomly generated sequence, created by ExPASy WWW server tool
PQEPSESESPQPSESEEEQPEQESPEQSSEEPSQEQEEQEEPSEEEEPEESPEPSEEQEPPPPEEPEESPPEPEEEEEEESESPEPQSESEEESPEEPPQSEEPQSPQPEPSPEEEPPEPEQPEPSPQSEEPQEPQEEEEPEEPEPEEEEPPEEESEESSQESPSEEPSSSPESEEEEEPPQEPSSESEPEEESPQEEEESEQSQESEEQQEESPSPESESSPPESQESESEEEEQESESSSQPSEPEEEQEEEEESPEPEQEPEPEESSSSSESQSESSEQESSQESEQSPPEEEESESSQESESPESEQEQPPEESEEEQPPEEPEEQPQEPQSSPQESPSSPESESPPSEPPPSEEEEPPEQEEPPESEEEPEEEEEEEEEPEEEEEEPSEESPESESEPPPPSSEPSEPSEPESPEEESSPEESQSPEEEEEESEEEPQPESSEPEEPEEQEQQEEQEEPPSPQPPEEQPQQQEQEQSEPSEQQEQPSSSPESEEESEPEEPEPEQESPPESEEESEQPPESPSSEPSSPEESQESSSPESPESPSPPESSQPEEEPQQEPEPSSPQPQEQPEEEESPPPSSPEQPEEPEEESSSQSSQEEQPSEEESEEEESQEEPSESSEEPEEEEEEPPESQSEEQSQEEQPESPQEEEQSESPPQPPEEPEEQSSQEESEEEQPSEQSSEEPSSESEESEPQESEEEEPPSEPESEQQSEEPPQSQEESPQPSPSEPEEEEQPSEEEPSQEQEPEEEEEEESSEPPEEEEPQEEPEEPPEEEEEEEQSEEEEEPEEPSEQEEEPPEEPEESESESPSPEPSSSEQSSPSEQEQSSEESQPEPEPEEQSEESSQPPEPEPPPPPESESSSSESESEQSESQEEPEPSEEPSEQSSESEEPESEEEEESPEEPEQEQPSEPEEPEPESEQEEESESPPPPPSEESPPQSSEPSPEEQPQESEPEPEPSSPPEPPPEEESSEPESEEESESSEQEPEEPPESESE

SEQ ID NO: 10 - protein, 1000 aa, Artificial Sequence
  Randomly generated sequence, created by ExPASy WWW server tool
EEEESSPPEEEESSPEPEEPEPEPSPPQEEEEEPSPQEQQPQQQESSQEEEQEPEEEEQESSSPQEEPPQPEEEPEPEEEEESSSEEEEPEEQEQPEPEEEPSPESSESESSSSEEEEEQPSQPESSPSEEEQPQEPEEPEPEEESPSPPEEQEEESESEEEQEQSEPEESEEEEEPSSPQSEQEEPQEPEPEEQEEEPPEEEEQEPPESESPEEQEEEQPPSPEEESEEEEEPEEEEEQEESEEEESQSPSEEPEPEESSSPESEEPPEEESSEESSEESQEESPSPEEEEESSESEQPPESPSESQESPSQSEEESQEEPPEEESSPEEEPPPSPSESEPPEEEEEPSESEEEEPPPEEEESSSEEQESEEPESEEESPEEQSEEEEESQESSPEPPEESPSEQPEPSPPEPESESSEPEEEEEEEEEPPSSEEEESEEPEQPEEEQEEPQEEEESPSEESPEEPEESEPEEESEEEEPEQQPEEEPPEEEEQESSEPSSPPSEEQSEEPEEQEEPPEPSQPEPQQESESSSPSESQPESQESEEEEEEEESEEESEPSQEPEEQQPEEEEEEEEEPEEEEEQSEPEESSEQQEPPQSSQPQEESEQEQEEPQSPEEESPPPEEEEPQEEPPEPEEEEPSEQPPSSPPEEQSEQPEQSEPQSESPSQPESSEQPEEQPEPPSPQSSEESEEPEEEEQSEEPSPSQSESSSSPEESEPPEEEEEEEEPEEPEQEEEQSEPQEQEPSEESSEPEEESSPSSQSSEQSSSEEESESEQSSPPPEEESPEEEEPEEEEPEESPEEESEESPESEESEESSEEQEESSPEEEPSEQEEPPEQEPESPPEQEEEEEQSEPQEEEPPESSEPEEESPPEEPQSEEEEEEPQPESESEPEEPSPEPESEESEEEPESESSSPPESSSEEEEEEPEEQSEEEEESQEEEEQEEEPSQEEEEPEEQQPPSEEEEQPEQSEEPEPSEPSEEEPEPEESPPESQPPSEEP

SEQ ID NO: 11 - protein, 1000 aa, Artificial Sequence
  Randomly generated sequence, created by ExPASy WWW server tool
GQQGSSPPSPSQGGQPPSSQPSQQSSSSPPPSPPPSSPPSQPPSPPSSGSGSSSPSQGSPPSPPSQGPPQPPQSPGSQGPPPPPGPGSGPPPSSSPQPSQPPPSQPSQQSPQPSPGPGSPSQQPSSGSQQSPGQGPQPQGPSGSPQGQGSPGSSSGPQPSSQGSPPGPPPGPSPSGGPQSSPGSPPSPQGSQPQSPGPSSPSSSPQPPSGPPSSGGQSSQGQSPSQGPPPGSPQPPGGSGPSPSSSPPPSPPPPQSSSSGSQQSSSSSGSPPSSSQGPPQSSSQPQSQSSPSQPPSGSPGSSSSPSPSPSGPSGSPSGPPSSPSGSPPPGGPPQSGGPGPSSGQQPPGPQPGSPPGQPQPGSSSQGPQQGPPPGSPQGPSQPGPQSPPSSGGSSSQPQSPSSGPGQPSPSPPGSPGGPGQPPSQPSPSSSSSQSGQSSQPSGPPSGQSQPGQPPQPSPPSPPPPSPPSQSGSGSPGPPSGPQPSSQPSPSQPGQGPSSSPPGQSGPSSPSSSQPPPSQSPPQSGQSPSSSPPQSSPSSGQQPSPGPPSSSSPQPSSSQGSPPPQPQGQSPPSQQPSQPGGSSQPSSPPPPGPQGPQPPSPQPPSGPGSQPQGGSPSSQGGQPSSSPPQSSSGPSGPGSSPSQSPSGQGPSSQPSPSGSGQPQGPPSPSGQPPSPPSGSPSPPQPGSPGQPQPSPPSQSPGGPGGPQGPPSSPGSSGSSGSSQPPPPPSQQSSSGQSPQPQGQGQQPGSPGQSGQQSQSPGGPSPQQPPPPPPPPPGSSPQSSPQPSPSQSQPQSGSQSSQQQSQSSSSPSPQSQGGPQSSGSSPSSGPQSPSPGGPPPSQSSSGQPSPPSPPGPSGSSSSSSGSGSGPQPSPPPQSPSQQSGSSQSSPSQSQPQPPPPGSGQPPPSGGPQQPPSPQQGSQSSSQPPPPQSSSSGGPGQSSGSPGPSPPQQSGGSPPPSGGGSGPGSPPSGQGSPSQSSGPSGGPGGSPPPPSSPSPSQSS

SEQ ID NO: 12 - DNA, 3000 bp, Artificial Sequence
  Sequence is produced using the reverse translation tool located at www.vivo.colostate.edu/molkit/rtranslate/index.htm l.
tctcaatctcctaaaccttcttctcaatctcaatctcaacctccttcttctaaaaaatctaaacaacaacaacaacctaaatctccttcttcttctcctcaatctcaatctccttcttctaaaccttcttcttcttctcctcaacaaccttctaaatcttctaaatctcctaaacctccttctccttctcctcctccttctaaaaaacctaaatctccttctaaaccttctcctaaacctccttctcctcctaaatctaaatctcctaaacaacctcaatcttcttctcaatctcaatcttcttcttctaaatcttctcaacctccttctcctccttcttctcaaaaaccttctcaatctcaatcttcttctcaacctaaaccttcttctcctaaacctcaatcttctcctcaaaaacaatctccttctcaacctaaaaaatctcaaaaacctaaaaaacaaaaaaaacctcaacaaccttcttctcctcaacctaaacctcaatctcaacctcaacctcctcaatcttcttcttctaaatcttctcctcaatcttctcaacaatcttctcaatctcctcctcctcctcctccttcttcttcttctcctcctaaatctaaaccttctaaacctcaatctcaaaaacctccttctccttcttctaaacctaaatctaaatcttctcctcaaaaatcttcttctccttctcctaaatctaaatctcctcaacctcctaaacaacaatctcctcctaaacctcctcctaaatctcctcaacctaaaccttctcctccttcttctcctaaaaaacctaaacctcctccttctcctaaatctcaatcttcttctcaaccttctcctaaatctaaatctcaacctccttcttcttctcaaccttctccttcttcttctcaacaatctcaatctcctcaaccttcttctcaaaaacctcctcaatctccttctcaaaaatctaaaaaatcttctcctccttctcctcctcctcctccttctcctccttctcaaaaacaacctcctcctccttcttctcctaaacctcctcctcaacaatctcctcaaaaatctcctaaatctcctaaacaatctaaacaatctcctccttctcaaccttctcctcctcctcctccttcttctcctcaacctaaaccttcttctcaacctaaacctcaatctaaacaacctcaacaaccttctaaatctaaacctcctcctcctcaatctaaacctcctcctcaatctccttctaaacctcaacaacaaccttctcctcctaaacctccttctaaacctaaacctcctcctcaacctaaatctaaatctaaaaaacctaaacaatctcctaaatctcctaaatctcctcctaaaaaatcttctcaaaaatcttcttctcctcctcaatctcctaaaaaacaaaaatctcaatctccttcttcttctcaacctcctaaacctcctaaacctccttcttctcctcctcctccttcttcttctaaacctccttctaaaaaacctcaatcttcttcttcttctccttctccttctcaacaacctcaaccttcttctccttctcaacctcctccttcttctcctcctcctcctcaaccttctcaacctccttctccttcttctaaaaaaaaacaaaaacaacctcaacaaaaacctcctcaacaacaatctcaaaaatctaaacaacaaaaacaacaaaaatcttctcctcctccttcttcttcttctccttctaaaaaacctcctcctccttcttctcctaaatctcaaaaaaaaaaacctccttctcaaccttctcctcaaccttcttcttctcaatctccttctcaacaatctcaatctaaaccttcttcttctcctcaaccttctcctcaacctaaatctcaatctcctcaatctcaaaaaccttctcctcaatcttctccttctaaatctaaacctccttcttcttcttctcaacctaaaccttcttctccttctcaacaaccttctcaacctcctaaatcttctaaatctaaacaacctcctcctccttctcaacaaccttctcctaaacaatcttcttcttctcctaaaaaaaaacctcctcaacctcctaaaaaacaatctcaacaaaaacctcctcctcaacctcctcctccttctcctcctcctcctcaacaaaaatcttcttcttctaaatctaaacaaaaatctaaaccttctccttctcaatcttctccttctcctccttctcctcctcctcctcaatctcctaaacaaaaatcttctaaatctcctcctaaacaaccttctcctcctcaacctcaatctcctaaaaaacaacctcaaaaatctcctccttctcaatctccttcttctcaatcttctcctcaaccttctcctcctccttcttcttctcaatctcctcctcctcctaaatcttctcaatcttcttcttcttcttctaaacctcctccttctcctaaacctcctcctcaaccttctcctcaatcttctcaacctcaaaaaaaatctcaaccttcttcttctaaatctcctaaacctcctcctccttcttctaaacctcctaaacaatcttctcctaaaccttctcaacctccttcttctcaatctaaacaacaaaaacaatctaaaaaaaaatctaaaaaaaaaccttctcctcctaaaaaatctaaacaacctcaacctcaatctccttctaaatctcctaaaaaaccttcttctaaatcttctaaatctcctcctaaatcttctccttcttctccttctaaatctcctcctcaaaaacctccttctcaaaaatcttctaaacctcctcctccttcttcttctcaatctaaacctcaacaatctcctaaaccttctaaaccttctcctccttcttcttcttctcctcctcaacaacaatcttcttcttctaaacaatctcaatctcctcctcctccttcttctccttctccttctccttct

SEQ ID NO: 13 - DNA, 3000 bp, Artificial Sequence
  Sequence is produced using the reverse translation tool located at www.vivo.colostate.edu/molkit/rtranslate/index.htm l.
aaacctcctcctaaatctcaaaaaaaatcttctaaaaaacctcaacaaaaatcttctaaatctcctaaatctaaaaaatcttctaaacctcaaaaacaaaaatctaaacctcctaaatctaaatctcaacctcctaaaaaatctaaacaaccttctaaaaaaaaaaaaccttctaaaaaacctcctaaatctaaacaacaaaaacctaaaaaaaaatctccttctcctcctcctcaatctccttcttctaaaaaaaaaccttcttcttctcctaaacctaaaaaaaaaccttctcctccttcttctaaatctaaaaaacctaaatctccttctccttctaaatctaaacaacaatctcctcaaaaatctccttctcctaaatctaaacaacaatcttctaaaaaatctccttcttcttctcaatctcctcctaaatctaaaaaatcttctaaaaaatcttctaaaaaatctccttctcaaaaaaaacaacctcaacctcaatcttctcctcctaaacctcctcaacctaaaccttctcctaaaccttcttcttctcctcctcctaaacctcaacaacctcctaaacctccttctcaaaaatctcctcctaaacctaaaccttcttctccttctcaaaaaaaatcttctcaaaaatctaaacaaaaacaacctcctcctccttcttctaaaccttctaaatctaaacctaaaaaaaaaaaatcttctcctaaacaacctcctccttctcctcaacaatcttctaaacctaaaaaatcttcttcttctcaaaaatctcctcctcaaaaacaacaaaaaccttcttctcaatcttcttctcctcctcctcaatctaaatctaaaaaatcttctcctaaaaaatctcctcctaaatctaaaccttctcaacctcaaccttcttcttctaaacctcctaaatctaaatcttctcaacaatcttcttcttctcaaaaaaaaccttctcaacaacaaccttcttctcctaaaaaacctcaatctcctccttctcctcctcctaaacctcctcctcctcaatcttcttcttctaaatctcctcctaaaaaatctaaatcttctcctaaacaacctccttctcctccttctcaatcttctcaacaatcttctaaatcttctccttctcctcctaaaaaaaaaaaacaacctaaacaatctaaacctaaacaacaaccttctaaacaatctaaaaaaaaacctcctcctcaacctaaaaaatctcctcaaaaacaaaaatctcaacctaaaaaacaacaacaaaaaccttctcctcaacctaaatcttcttctaaatcttctaaaccttcttctcctaaaaaaaaacctcaatcttctcctcctcaacaaaaacaaccttctaaacctcctcaatctccttctcctcaaaaatctcaaaaatctcctcaacctccttctcctcctaaatctcctcaacctcctaaaaaatctaaatcttcttcttctaaatctaaaaaatcttcttctcaaaaacctcctcctcaacctaaaccttctcaacctaaatctcctccttctcaatctaaaaaaccttctaaacctccttctcctccttctaaacctaaacaacctcaatctcctaaatctaaacaacaatcttctcctccttcttctccttctaaatctaaacaaaaacctcctaaacaatcttctcaaccttctcaacctcctcctaaatctccttctccttcttctcctaaatctaaacctaaacctaaaccttctcaatcttctaaatcttctaaaaaaaaaccttctaaacctccttctcaatctccttctcaaaaaaaatcttctaaatctcctcctcctaaatctaaacctcctccttctcaatctcctaaatctaaaaaaaaatctccttctcaaaaatctaaaaaaaaaaaacaaaaaaaacctaaacctaaacctcctccttctcaaaaaaaacaacaaaaatcttcttctcctcctccttctaaaaaatcttctccttctaaatctaaacctccttctcctccttctaaaaaatcttctaaatctcctcctcctaaaaaaaaacctcctcctcaatctccttctcctaaacaatctcctcaacctaaaaaaccttctaaatcttctcctcctcaacaatctcctaaaaaaaaatctcctaaacaacctccttctaaacctaaacctaaacctcctcctaaacaaaaaccttcttctaaacctcaaaaatcttcttctaaatctaaaaaacctaaacctccttctaaacaatctcaaaaaaaatctaaacaacctcaatctcctcaaccttcttctaaacaaaaacctaaacctaaacaatcttctcctcctaaatctaaatctaaaaaaaaacctcctcaaaaaaaaccttctcaacctaaatcttctaaaccttcttctaaacctaaaaaaaaacaacctcctcctcctcaacctaaacctcctcaaaaaaaatctaaacaatcttctaaatctcctcctcctccttctaaaaaatctaaaccttctaaaaaatctcaacaacaaaaatctcaatctccttctcctaaatcttctcctccttctcctaaacctaaaaaatctcctcctccttcttcttctccttcttcttctccttcttctcctaaacctccttcttctcaatctcaaaaaaaacaatctcctaaacaacaaccttctaaacaaaaatcttctcctcctaaaaaatctaaaaaacctaaaaaacctcctccttctccttcttctaaaaaaaaaaaacctaaaaaatctaaatctaaaaaacctccttctcctaaacaaaaaaaatctaaacaaaaatctaaacctaaacctcctaaacaacctcaatcttctcaacctcctaaacaacctaaacctcaacaacaatctcaatcttctcaacctcctcaacaatctcaaaaacctcaaaaacctaaatctcctcaacaatct

SEQ ID NO: 14 - DNA, 3000 bp, Artificial Sequence
  Sequence is produced using the reverse translation tool located at www.vivo.colostate.edu/molkit/rtranslate/index.htm l.
caatcttcttctcctcctaaatcttcttctcaatctaaatcttcttcttcttcttcttcttctccttctcctaaatctccttcttctccttctaaacctcctcctccttctaaaaaaaaacctaaatctaaaaaaaaacaatcttctcctaaatcttctaaacctaaaaaacctaaacaaaaaaaatctcctcctcctcaaaaacctaaaaaatctccttctaaacctaaatctaaaccttcttcttctaaaaaaaaaaaatctcaacaacaatcttctcaaaaatctcaatctaaacaacctaaaaaacctcaaccttctcctaaaaaacctaaatctcctaaaaaacctcctaaacctcaacctaaatcttctcctaaacaatctaaacaaaaaccttctaaaaaaaaaccttcttctaaacctaaatctaaatctaaaaaaaaatctcaaaaacctaaacaatctaaaaaatcttcttctaaacctccttctaaatctaaaaaaaaacaacctaaacctaaaaaaaaatctaaatcttcttcttctaaatcttctaaatctccttctaaatctaaatctcctcaatcttctaaatcttctcctcctaaaaaacctaaacctaaaaaacctaaacctaaatcttctaaatctcctaaatctcctcctaaaaaaaaacctcaatctcaaaaacaacctaaatctcaatctcctcaacctcaaaaaaaacctaaacaatcttctaaacaaaaacctaaatctaaaaaatctcctaaaaaacctcctaaaaaatctaaacctaaatctcctcctcctcctaaaaaacctaaacctaaaaaatcttctaaacaacctaaatctcaatcttctcaaaaaaaacctaaacctcctcctccttctcctcctaaacaaaaacctcaaaaatcttcttctcctcctaaacaacaatctaaaaaaccttctcctcctcaaaaacctaaacctaaatcttctccttctccttctaaatcttctcaatctaaaaaaaaaaaacctaaaaaacctaaacaatctcctcctcaaaaacctccttctaaacaatctcctcaaaaacctaaatcttcttctcctcctaaaaaaaaaaaatcttctaaaaaacaaaaaaaaaaacaaaaaaaacaaaaatcttctcaatctaaaccttctcaaaaacctccttctaaacctaaatcttcttcttctaaaaaaaaacaatctaaaaaaaaaaaacctcctcaaaaatcttctaaaaaacaacaatctcctcctaaacaatctcctaaaccttctcctaaaaaaaaaaaacctaaaaaaaaacaaaaaaaatctcctaaacaatctcaacctaaaaaacctaaaccttctaaacctcaaaaatctcaaaaaaaatctccttctcctaaacctcctcctcaacctaaacctcaaaaaaaatctcctcctaaacctaaacctaaatctccttctcctcctccttctcaaaaacctaaaaaaccttctaaacctcaacaatctcctcaaaaaaaacctcctcctaaatctcaaaaaaaacctaaacctcctaaaaaaaaatctaaatcttcttctcctcctcaatctaaacaacaaaaaaaaaaaaaaaaaaaatctcctaaatctaaaaaatctaaacaacctcaacctaaacaaaaaaaaaaatctaaacctaaatctccttctcaaaaacctaaacaatcttcttctaaacaaaaaaaatctcctaaacctaaaccttctcctaaatcttctaaacctcaacctaaaaaaaaaaaaaaaccttctaaaaaaaaaaaaaaaaaaaaacaaaaacctcctcctcaatctaaaaaacctaaatctcctcctcctaaacctaaacctaaatcttcttctaaaaaacctcctcctaaaccttctaaacctcaatctaaaaaacaatctaaatctaaaaaaaaacctcctaaacaaaaaaaaaaacctaaaaaatctcctaaaaaaaaaaaaaaacctccttcttctaaatcttctcctaaatctcctccttctcaacaatctcctcctcctcctaaacaatctaaacaacctccttctcaatctaaaaaacctcctaaacctcctaaaaaaaaatcttctaaaaaaaaaaaaaaatctaaaaaacctcaaaaacaacctaaaaaaaaatcttcttctaaacaatctaaatctaaacctccttctccttctcaacctccttctccttctaaacctccttctcctaaaaaaaaatctccttctcaatctaaacctaaacaaaaatctccttctaaatcttctaaatctaaacaatctaaaccttctaaacaacaacctaaacaaaaacctcaatcttctcaaaaacctaaatctcctaaatctaaaaaaaaatctcaaaaaaaacaatcttcttctcctcctaaatctaaatctcaacaacctaaaccttctcaaaaaaaacctcctaaacaacaatcttctaaatctcctcaaaaatcttctaaacaaaaaccttctaaaccttcttctcctaaacctcaatctaaacaatctaaacaacaaaaaaaaaaaaaacaatctaaacaacctcctaaacaaaaaaaaccttctaaatctaaaaaacctcctcctaaacctcctcctaaatctaaacctaaacaaaaaaaacctcaaaaaaaacctaaatcttctaaaaaacctcaacaaccttctccttcttctccttcttctaaatcttctaaaaaatctaaatctaaacaaaaacctcctcctcaacctcctccttctcaaaaaaaaaaaaaacctcctcctaaatctcaaaaaaaacctaaaaaaaaaaaatcttctccttctaaaaaaaaacctcctaaaaaaaaatctccttctcaatcttctcaaaaatctaaatcttcttctcaatctcctcctcaacaacctcctcaaaaacctaaaaaatctaaacaaaaaaaaaaa

SEQ ID NO: 15 - DNA, 3000 bp, Artificial Sequence
  Sequence is produced using the reverse translation tool located at www.vivo.colostate.edu/molkit/rtranslate/index.htm l.
tcttctaaacctaaaaaatctcctccttctaaaaaacaatctcaatctaaaaaatctaaacctaaaaaaaaaaaatctcaaaaacctaaaaaatcttctcctaaaaaaaaatctaaatcttctaaaaaaccttctcctcctcaaccttctaaacaacctaaacaacaatctccttctaaacaatctaaatctcctaaatctcaaaaacctccttctcctcctaaaaaaaaacaaaaaaaaccttctaaacaacctaaatctcctaaacctcctaaatctaaatctcaacaacctaaacctaaacctcaacaacctaaaaaaaaacctaaaccttctaaacctcctcctccttcttctcaaaaacaacaaaaatctaaatctccttctcaaaaaaaaaaaaaaccttctaaaaaacctaaaaaaaaacaacctaaacaatctccttcttctaaaccttcttctcaacctaaacaacctcctcaaaaaaaaaaaaaacctaaacctaaaaaaaaaaaaaaacaaaaacaacctaaaaaacctaaaaaaaaaaaatctcctaaaaaaaaacctaaacctcctaaatctaaaaaaaaaaaacctaaatcttctaaaaaatctaaacctcaaaaaccttctcctcctaaatctcctaaacctaaacctaaacctaaaaaaaaacctaaatctaaaaaatctaaatcttctaaacctaaacctccttctaaaaaaaaacctcctccttctcctccttcttctcctaaacaaaaatctaaatctcctcctaaaaaaaaacctaaacaaaaacctaaacaaaaatctaaatcttcttctcctcaacctaaacctccttcttctcctaaaaaaaaaaaaaaacaatctaaatctaaaaaaccttctaaaaaatctcctcctaaaaaaaaaaaatctcaacaaaaatcttctaaaaaacctaaaaaacctaaaaaatctaaaaaatcttctaaaaaaaaatctaaacctcaatctaaacctaaatcttctaaaaaaaaaaaatcttcttctaaatcttctcctaaaaaacctaaacctcaacaacctaaaaaaaaaaaacaacaaaaaaaaaaaaaatcttctaaacctaaacaaaaaaaatctcaaaaaaaaccttctaaaaaaaaacctaaaaaacctaaacaaaaaaaatctaaaaaatctcctcctaaaaaacaatctaaacaacctcctcaaaaaaaatctaaaaaaaaacaaaaacctccttctcaaaaaaaatctcaatcttctcctaaacctaaacctcctcaaaaacctaaaaaaaaatctcctaaacctcctaaaaaacctcaaaaaaaacctaaatctaaacaatcttcttctaaaccttctaaacctcctcctcctaaaaaacctcctaaaaaacctaaacctaaaaaaaaaaaaaaaaaatctaaaaaatcttctaaaaaaaaaaaacaaccttctcctaaaaaacctaaatctaaaaaaaaaaaaaaatcttctaaaccttctaaaccttctcaacaaaaatctcctaaatctaaaccttcttcttctcctcaatctaaacaacctaaacaatcttcttcttcttctaaaaaacctaaaaaacctccttctaaatctaaacaaccttcttctaaatctcctaaatctcctcctcctaaaccttctcaaaaacctcctcctcaaaaaaaacctaaacaaaaaaaatctaaaaaacctcctaaaaaaaaaaaaaaacctcaaaaacctaaaaaatcttctccttctcctcctccttctcctaaacaaaaaaaaaaacaacctccttctaaacaacctaaatctaaaaaatcttctcaaaaaaaatcttctaaatctaaaaaaaaaaaaaaaaaaaaacctcctaaaaaatctaaatctcctccttctcaatctaaatctaaaccttctcctcctcctaaaaaacctaaaaaacaatcttctcaacaatctaaatctcaacaatcttctaaacctaaacctaaacctaaaaaacctcctcctaaacaatctccttctccttcttctcaaaaaaaaaaaaaacctaaatctaaaaaaccttcttctccttcttctcctaaatcttcttctccttcttcttctccttctaaatcttctaaacaaaaaccttcttctccttctaaacctaaaaaacctaaaaaaaaacctaaaaaaaaacctaaaaaacctaaaaaacaacctaaacaaaaacctaaaaaacctcctccttctaaaaaacctaaacctccttctaaatctcaatctaaaaaacctaaacaaaaaaaatcttctcctaaaaaaaaaaaatctaaaaaatctaaaaaatctaaacaacaaaaacaacaaaaaaaaaaatctcaaaaaaaatctaaatcttctcctcctaaatctaaaaaacaaaaacaatctaaaaaacctaaacaacctaaaaaaaaacaatctaaatctcctaaaaaacaaaaaaaacctaaatcttctccttctcaaaaacaacaacaaaaaaaaaaaaaacaaccttctaaatcttctaaaaaacctaaacaaaaaaaaaaatctaaacaatctaaacctaaacaacctaaaaaatcttctcctcctaaatctccttctaaacaatctaaaaaatctccttctaaatctcaaaaacctcaatctaaaaaatctcctaaatctaaaaaaaaatcttctaaaaaaaaaaaaaaaaaaaaaaaacctaaaaaacctaaaaaaaaacctaaaaaatctaaatcttcttctcaaaaaaaatctaaacaacctaaatctccttctcaaaaatcttctaaaaaaaaaaaacctaaacaatcttctaaaaaaaaacaaaaaaaacaaaaacaaaaaaaaaaacaaccttcttctaaacctcaacctaaaaaaaaacaacctaaaaaaaaacaaaaaaaacctaaaaaaaaaaaatctcctaaatctcctaaacctaaa

SEQ ID NO: 16 - DNA, 3000 bp, Artificial Sequence
  Sequence is produced using the reverse translation tool located at www.vivo.colostate.edu/molkit/rtranslate/index.htm l.
aaaaaaaaacaacctaaaaaatctcaacaaaaaaaaaaaaaaaaaaaacaatctaaacctaaacaaaaaaaacctccttcttctaaacctcctaaacaaaaaaaaaaacaacctaaaaaatctccttctaaatcttcttctaaaaaaaaacaaaaatctcctaaacctcaaaaaaaacctaaaaaacctaaaaaacctaaaaaatctaaaaaacaacctcaacaacctccttctaaaccttctcctcaatctaaatctaaacaacctcaacaaaaaaaacctcctaaacctaaacctcctaaaaaacctaaaaaaaaaaaacaaccttctcaaaaacaatctaaacctcctaaatctcaatctcaaaaaaaatcttctaaacaaaaatctccttctaaacctaaacaaaaatcttctaaaaaaaaaaaaaaaaaaccttcttcttctccttctaaatctaaaaaaaaaaaacctaaatctaaacctcctaaaaaatctaaacctaaaaaaaaaaaaaaatctcaatctaaaaaacctaaaaaaaaaaaacctaaacaacaacaaaaacctaaaccttctaaacaacaaaaacctaaaccttcttctaaaaaatcttctcctaaaaaaaaacctaaacaaaaacctaaacctcaacctaaacctaaaaaacctaaacctcctaaacctaaacaaaaaaaaaaatctaaacctaaacctaaatctcctaaaaaaaaacaacaacaacaacctaaacctcctcaaaaatctcctaaaaaatctcctcctaaaaaacctaaacctaaaaaatcttctccttctaaatctccttctaaacctaaaaaacaaaaacctaaaaaaccttcttctcaaaaaaaacctaaatctaaatctcctcctaaaaaacaatctaaaaaatctaaatctaaatctaaaaaaaaatctccttcttctaaaaaatctaaacctaaaaaatcttctcctaaaaaacctaaatctaaaaaacaatctaaatctaaatctcaaaaacctaaatctaaacaatcttctcctaaacaaaaaaaaaaatctcaaaaatctaaacctcaaaaatctaaaaaaaaatcttctcctaaaaaacaaaaatctaaaaaaaaaaaatctcctaaaaaaccttctaaacctcctaaaaaaaaacctcctaaatctaaacaatctaaaaaaaaacaatctcctaaacctaaacctccttctccttctcctaaacctaaaaaaaaatctaaaaaaaaaaaaaaaaaacaaccttcttctaaaaaacaacctaaaaaaccttctaaaaaaaaaaaacaatctccttctaaacaacctaaatctaaatcttctaaaaaaaaacctcctaaaaaacaacctaaaaaacctaaaaaaaaaaaacaatcttctaaaaaacctaaaaaatctcctcaaaaaaaatctaaaaaacctcaatcttctcctaaaaaatctccttctaaacaacctaaaaaaaaaaaacctaaaaaacctaaaaaacctaaaaaaaaaaaacctcaatcttctccttctaaacctcctcctaaatctcaatctaaacaaaaatctcctcctaaatcttcttctaaaaaaaaacaaaaaaaacctaaacctaaaaaaaaaaaaaaaccttctaaaaaaaaacctcctccttctaaaaaacctaaaaaatctaaaaaatctaaatctaaaaaaaaatctaaaaaaaaatctcctcctaaaaaatctaaaaaaaaacaacctaaacctcctaaaaaatctaaaaaaaaatcttctaaacaatctaaacctaaaaaatctcctaaacctaaatctaaaaaaaaatctaaaaaacaaaaatcttcttctaaaaaatctcctcctcctaaatctaaacctcctaaaccttctcaacctcctaaatctaaaaaaaaaaaacctccttctaaaaaaaaacctaaaaaacaaaaatcttctcaaaaacctaaatcttctcaaaaaaaaaaacctcctaaacctaaaaaacaacctaaatctaaaaaacctaaaaaacctaaaaaacaacaacaaaaaaaacctcctaaaaaaaaaaaaaaaaaaaaaaaaaaaaaacctaaacctaaaaaacctcctaaacctcaatctaaatctaaaaaaaaaaaaaaatctcctccttctcctccttctcctaaaaaaaaaaaaaaacaaaaaaaaaaatctaaaaaaaaaaaacctaaaaaaaaacctcaaaaaaaatcttctaaacaaaaaaaaaaaaaaccttcttcttctaaacctaaatctcaatctaaaaaatcttctaaaaaacctaaacaatctaaacaaaaaaaatctcaatctaaaaaatcttcttctaaatctaaacctcaaaaaaaatctaaaaaaaaaaaaaaaaaaaaacctaaaaaaaaaaaaaaaaaaaaatctaaatctaaatcttctcaatctcaaaaaaaaaaaaaaaaatctcctaaaaaaaaaaaaaaaaaatctaaaaaaaaaaaatctaaaaaacctcctaaacctaaaaaacaatctaaaaaatctaaatctaaacctcctccttctaaacctaaatcttctaaatctaaacctaaaaaacctcctaaaaaaaaaaaacaaaaaaaaaaacaaaaatctaaaccttctaaaaaatctccttctaaacctccttctaaaccttctaaacaaaaaaaaaaatctcaaaaaaaacaacctcaacctcctaaaaaacaacctcctaaatctaaacctaaacctcctaaacctcaaaaatcttctaaaaaaaaaaaaaaaccttctaaaaaacctcctaaaaaaaaatctaaaaaacaaaaaaaaaaaaaatctcaatctcaaaaaaaatcttcttctcaaaaacctaaatcttctaaatcttctcaaaaaaaacctaaaaaaaaatctaaatcttctaaacaaaaatctaaaaaacaaaaatctaaaaaaaaacctaaa

SEQ ID NO: 17 - DNA, 3000 bp, Artificial Sequence
  Sequence is produced using the reverse translation tool located at www.vivo.colostate.edu/molkit/rtranslate/index.htm l.
gaagaaccttctccttctcctcctgaatcttcttctgaacctcctcctcctcctcctcctcaacctcctgaacctcctcaacaatctgaacaacctcaagaatcttctccttctcaatctcaatctgaaccttctgaacaacaacaagaatcttcttcttctgaacaagaatcttcttctcctcctgaatctcaagaagaacctcaatctgaacaaccttcttctcctcctgaacctcaacctcaatctcaatcttctcaacctcctccttctgaatctccttctcaacaatctgaacctcctcctgaacaatctcaatctccttcttctccttcttcttcttctcaacaatctcaacctccttcttctgaaccttctgaaccttctccttcttctcctcaatcttctccttctccttctcctcaacaatctcctgaagaatctgaatctcaacctcaatctccttcttctcaatctcctcctcaacctccttctgaaccttctcctcctcaatcttctgaacctcctgaacctccttcttctgaacctcaaccttctccttcttctcctcctcaacctgaatctccttcttcttcttcttctcctccttctcctccttctcctcaagaaccttctcctgaacaacctcctcctcctcctcctcctcaatctcctgaatctcctccttctgaacctcctcaatctcctcctgaacaagaacctgaacaacctcctgaacctgaatcttctcctcctcaatctcaatcttctgaacctcaatctcaacctgaacctcaatcttctgaacaatctgaagaatctgaatctcaacaagaacctccttcttctcctgaacctccttctcctgaagaagaacaaccttctccttcttctccttctcctcctcaatctcctcctgaacctcctccttcttctgaacctgaatcttctccttcttctgaatctccttctgaacaatctcctcctgaaccttctgaacaatcttctcaatctccttctccttctcctcctcaacaagaacaatctcctccttctcaatcttctcctgaacctccttcttctcctgaacctgaagaatctcctcctcctgaacctgaatcttcttcttctccttcttcttctcaacctgaagaacaaccttcttctccttctcctccttctcctccttcttcttctcaatcttctccttcttctcaatctccttcttctcctgaagaatctccttctcctcctcctcctcctcctgaatctgaaccttctcctcaacaaccttctcctcctcaacaagaacctcctccttctcaatcttctccttctcaacaatctcctcctcctccttcttctcctcctccttctgaacaacctcctcaagaacctcaacctccttctcaatcttctcaacctcctgaaccttcttctcaatctgaaccttctcctcctcctcaatctcctcctcaacctgaatctcctcaaccttcttcttcttctcaaccttcttctgaacctccttctccttcttcttctcctcctgaaccttctccttctcctgaacaacctcctccttctccttctcaagaagaaccttctcaagaaccttctcaatctgaatcttctgaacaatctcaatctcctccttctccttctgaatcttctcaatctcctcctcaatcttcttcttctcctcaatctcctgaacctcaacctcctccttctgaatctcaagaatctcaacctcctccttctgaatctcaaccttctcctgaagaatcttctccttcttctcaatctgaacaaccttctcaatctcaagaacctcaacaatctcctcctcaaccttctcctgaacaacctgaatctgaacaagaatctccttctccttctgaagaatctgaatcttcttcttctcaatctcctcctccttctcctcaagaaccttctcctccttctgaatctcaatcttctccttcttctcctcctcaaccttcttcttctcaagaatctccttcttctcaacctcaacctcaatctcaatctcctcctcaacaacctcaacaatctcctcctccttctcctcctcctcaacaatctgaagaacaagaacaagaatctgaacctcaagaacctcaacctcaatcttctcctgaatctccttcttctgaatctgaatctgaatcttctcctgaacaacctcctcaacctcctccttctcctgaacctcctcctccttctccttctccttctcctccttctgaatctcaaccttctcaacctcaaccttcttcttcttctgaatctcctgaagaatctcctcaacctcctcctgaagaatctccttcttcttcttcttctgaagaacctcctcaacctgaagaagaacaatcttctgaaccttcttctcaatctccttcttcttctccttctccttctcaatctgaatctcaatctcaatcttcttctgaatcttcttcttctgaatctgaatctcaatctcctgaacctgaagaacctgaacctccttctcaagaatctcctcctgaacaacctcaacaagaacaacaacctgaagaatcttcttcttcttcttcttctcctcaatctgaacctcctgaagaaccttctcctcaacaacaacaatcttcttcttcttctcctgaatcttctcctcctcctgaacaagaacaacctgaacaatctcctcaacctccttctcaatctcctcaatcttcttctcaagaatcttctgaacctcaacctgaacaacaatctcctgaagaagaaccttctccttctcaatcttcttcttcttctccttctcctcctcctcctgaacaatctgaacaacctgaacctcctgaatctcctgaacctcaacaacaatctcctcaacctccttcttctcaagaacctgaagaacctgaacctcaatctcctcctgaatctgaacctcctgaagaagaatctcaatctcctcaacctcaa

SEQ ID NO: 18 - DNA, 3000 bp, Artificial Sequence
  Sequence is produced using the reverse translation tool located at www.vivo.colostate.edu/molkit/rtranslate/index.htm l.
gaacaacctgaacctccttctgaatctccttctccttctcctccttcttctgaatcttctcctcctccttcttctgaaccttcttctcctcaatctcaatctcctgaagaagaaccttctcaatctcaaccttctgaatcttctcctgaaccttctcctgaacaatcttctccttctgaagaagaacaacctcctgaatcttctcaatctcaagaatctcaagaacctcctgaatctcctcctcaacaaccttctcctccttctcaagaatcttctgaacaagaatctcctgaacaagaagaatctgaacctccttctgaagaacctgaacctccttctgaatcttctgaagaagaacaagaacaatctcctcaatctccttcttctgaacctgaacctgaacaatctcaagaatctccttcttcttctgaatctccttctcctgaagaatctcctcctcaacctcctgaacctcctgaatctcctcctccttctcctgaacaagaacaacaacctgaagaagaatctcctcctcaacctgaatcttctccttctgaatcttcttctcctgaatctcctcaagaacctccttcttctcctcctcctgaatcttctgaagaagaagaatctcaagaatcttctcctcaacaatctgaagaacaatcttcttctccttctccttctcaatctgaatctcaacaagaatctcctgaacctccttctcaacctccttcttcttctgaaccttcttctccttctccttctcctgaacctgaacctcaacaacctcaacaacaatctcaacctgaatctccttctccttctcctcaacaaccttctcaaccttctgaagaatctcctgaatctcctgaacctccttcttctgaaccttctgaaccttctgaagaacctgaatctgaacaagaaccttcttctcctcctgaatcttctgaacctgaacaatctcaagaagaacctgaacctgaacaatctcaatctgaatcttctcctgaagaatctcctgaatcttctgaacaacaacaagaacctgaacctccttctccttcttctcaatctcctccttcttctcctccttcttctgaacctccttctcctcctgaaccttctccttcttctgaatctcctgaacaacaacaagaagaacaaccttctgaagaacctcaatcttcttctgaagaacaatctcaatcttctgaacctcctgaaccttctcctcaatcttctccttctcctcaatctgaacctcctgaacaagaacaagaagaacctgaacaatctgaacctcaacctgaacctcctgaacaatctcctgaaccttcttcttctcctgaacaacaacctgaacctcctcctcaatcttcttctcctccttctcaagaagaatcttctcctcctgaagaatcttctcctgaagaatcttctgaagaaccttcttctgaacaacaacaagaaccttcttctcctcaagaacctgaaccttcttctcaacctcctgaacctcctcaacaacctgaacctgaaccttctgaacctcctccttctcaatctgaacctcctccttctcctcctgaagaacaacaatcttctcctcctgaacctgaacctcctcctgaatctccttctcaagaagaacctccttcttcttctcaagaagaacaacaagaacctgaatctcaagaacctgaagaatctcaacctgaacctccttctcctcctcaacctgaagaagaatctcctcaatctgaagaacctccttctccttctcaaccttctccttctgaagaacaatctgaaccttctcaacaacaagaaccttctcaaccttctgaatctcctgaatctcctcaagaatctgaacaagaacctgaagaacctgaatcttctcctgaagaagaatctccttctcctcaatctcctccttcttctcctcctcctgaatctgaagaacaacctgaagaacaacctcctcaacaatctcctgaacctcctccttcttctcctgaatctcctgaatctgaacctgaagaatctcctcctgaagaatctgaagaacaacctcaacaaccttctcaagaagaacctcctgaatctcaagaatcttcttctcctcaatcttcttctgaagaatctcctcctcctcaagaatctgaacaacctgaacctgaatctgaacaagaacctcctcctgaacaacaacctgaacaatctgaacaatcttctgaacaacaacctcctcctgaatcttctcaacctccttcttcttcttctgaatctgaagaagaagaagaatcttctgaacaagaaccttcttcttctgaagaacctgaatcttctgaatcttcttctgaacaatcttctgaatctgaagaatctgaagaagaacctcctcaacaacaagaagaatctcctccttctgaagaagaagaacaacaacaacctcctcctgaacctgaatctgaatctcctgaacaatctcaaccttctgaaccttctccttcttctgaatctcaagaagaacctcaagaaccttcttcttctccttctcctgaagaacctcaagaagaatctgaagaatctcctcctgaatctcctgaatcttctcaaccttctccttcttctcaagaacctcctgaatctgaagaatctcaacctgaacaagaatcttctcctgaagaacctgaacctcctcctcctgaacctgaagaacctcctcctcctccttctcctgaacctgaagaagaagaacaacctcaaccttctcaacaatcttcttctcaagaagaagaatctgaatcttctgaagaaccttcttctgaaccttcttctgaacctgaagaatcttcttcttcttctccttcttctgaacaacaatctgaatctcaagaagaacctgaagaagaatctgaagaacctcctccttcttctgaatctcctgaagaagaagaagaaccttctgaacctcctgaatcttctgaacct

SEQ ID NO: 19 - DNA, 3000 bp, Artificial Sequence
  Sequence is produced using the reverse translation tool located at www.vivo.colostate.edu/molkit/rtranslate/index.htm l.
tctcctgaacaacctgaacctcaacctgaacctgaacaagaatctgaacctgaaccttctgaacctcctccttctcaagaagaagaatctgaagaagaagaacaatctgaacaacctgaagaagaatcttctgaaccttctcctgaatcttctccttctcctcaagaaccttctcctcaacaagaacctccttctgaacctcaacaagaatctgaaccttctcaatctccttcttctgaatctgaacaatctgaagaacaagaacctcaagaagaatctgaatctgaagaatctcctgaatcttctccttcttctgaaccttctgaagaagaatctgaacaatctgaatcttctgaagaagaagaacctccttctcctccttctcctgaagaagaatctcctgaatctcaagaacaacaagaacctgaacaacaatctgaacctgaagaagaatcttcttcttctccttctcctgaaccttctgaagaacctcctcctgaatctgaaccttctgaagaatctcctccttctgaacaatctgaacctgaacctcctcctgaatcttctgaacctcctcaacaagaacaagaatctgaagaatcttcttctcctcctgaatctgaacctcctgaacaatcttctgaacctgaagaagaacaacaatctgaagaagaagaatctcctgaagaagaatcttctgaagaatcttctcctgaacaatcttcttcttcttctgaagaagaatcttctgaagaacctgaatctcctgaagaagaagaaccttctcaacctgaacaacctcaacaatctcctcctcaagaatctcctcctgaagaatctcaagaacctccttctgaatcttcttcttctgaacaatcttctgaatctcaatctcaatctccttcttcttcttctgaacctcaagaacctcaacctcctgaaccttcttctcaagaagaacctgaacctcctgaacaagaacctgaaccttctcaaccttctgaagaatcttctccttcttctgaacctgaagaatctcctcctgaagaagaatctgaatcttctgaatctgaagaatctgaagaagaagaagaagaagaagaatctccttctccttctcctcaagaaccttcttctcaacctccttctgaagaaccttctgaagaaccttctcctgaagaacaagaatctgaagaagaagaatctccttcttcttctgaacaagaagaaccttctcaatctgaacaacaatctcctccttcttctcctcctgaatctgaacaatctcaagaagaagaacctgaagaagaagaacaacctcctgaaccttctcaatctcctgaagaatctgaatctgaagaacaacaatcttctgaatctgaacctcctcaatctcctcctgaagaacctgaacctgaacaacaacaatcttcttctgaagaatctgaacaagaatctgaaccttctcaagaagaatctgaatctgaatctgaagaatctgaagaatcttctccttcttcttctcctcaacctgaagaacctgaatctgaagaagaacaaccttctccttctcctgaatctcaagaacctgaagaatctgaaccttctgaagaaccttctcaatctcctgaagaagaagaagaagaacctgaacctgaacctcaacaatctgaagaagaacaacctcaagaatcttctcaacaagaagaagaagaacctcctgaatctgaacaacaaccttcttctgaacaagaagaatctgaagaacctcaacaagaagaaccttctgaatctcaacctcaacctcctgaatcttctcctccttctcctcctcctcctgaagaaccttctcaagaagaatctgaacaagaacctgaagaagaacaatctcctcctgaacctgaagaacaagaaccttctccttctgaatctgaagaatctcctcctgaatctgaatcttctgaagaacaacaagaagaatctgaacctgaatctgaagaagaacctcctcaacaatctgaagaacaacaatctcaacctgaagaagaagaagaagaacaatctgaagaaccttcttcttctcctcctgaacctcctcaacaagaaccttcttctccttctgaacaacctcctcaacctgaagaacctgaacctgaagaagaatctgaagaaccttctcctgaacaaccttctgaatcttctgaacctcctgaatctcctgaagaaccttctcctcctcctccttcttctgaagaatctgaatctgaatctgaacaacctgaagaacaacctgaatctgaagaacctccttcttctccttctgaatcttctgaagaacctgaagaagaacctgaagaagaacaaccttctgaacctcaacctccttctgaacaaccttctcctcctgaagaacctcaagaagaatctgaagaagaacctccttctgaagaaccttctcaatctgaatctcctgaacctgaaccttctccttcttctcctcctcctcaagaacctgaacaaccttcttcttctgaacaatctcctcctgaaccttctgaacaatctcctccttctcaagaagaacctgaagaagaaccttctcaatctgaacaagaatctgaagaacaacctcaagaagaacctcctcaaccttctcctgaaccttctcctcaagaaccttctgaacctgaacctgaagaacctcctgaagaagaacctcctcaacctcctccttcttctgaacctgaagaacaagaatcttcttctcctgaacctcaacaacctcaaccttcttcttctcctgaagaagaacctcctgaagaatctcctgaaccttctcctcaacctgaacctgaatctgaacctgaagaagaacaatctccttctgaacaagaacctgaagaagaagaatctcaagaaccttcttctcctcaagaacctgaagaagaacaatctgaatctgaatctccttctcctgaacctgaacctgaacctgaagaa

SEQ ID NO: 20 - DNA, 3000 bp, Artificial Sequence
  Sequence was produced using the reverse translation tool located at www.vivo.colostate.edu/molkit/rtranslate/index.htm l.
cctcaagaaccttctgaatctgaatctcctcaaccttctgaatctgaagaagaacaacctgaacaagaatctcctgaacaatcttctgaagaaccttctcaagaacaagaagaacaagaagaaccttctgaagaagaagaacctgaagaatctcctgaaccttctgaagaacaagaacctcctcctcctgaagaacctgaagaatctcctcctgaacctgaagaagaagaagaagaagaatctgaatctcctgaacctcaatctgaatctgaagaagaatctcctgaagaacctcctcaatctgaagaacctcaatctcctcaacctgaaccttctcctgaagaagaacctcctgaacctgaacaacctgaaccttctcctcaatctgaagaacctcaagaacctcaagaagaagaagaacctgaagaacctgaacctgaagaagaagaacctcctgaagaagaatctgaagaatcttctcaagaatctccttctgaagaaccttcttcttctcctgaatctgaagaagaagaagaacctcctcaagaaccttcttctgaatctgaacctgaagaagaatctcctcaagaagaagaagaatctgaacaatctcaagaatctgaagaacaacaagaagaatctccttctcctgaatctgaatcttctcctcctgaatctcaagaatctgaatctgaagaagaagaacaagaatctgaatcttcttctcaaccttctgaacctgaagaagaacaagaagaagaagaagaatctcctgaacctgaacaagaacctgaacctgaagaatcttcttcttcttctgaatctcaatctgaatcttctgaacaagaatcttctcaagaatctgaacaatctcctcctgaagaagaagaatctgaatcttctcaagaatctgaatctcctgaatctgaacaagaacaacctcctgaagaatctgaagaagaacaacctcctgaagaacctgaagaacaacctcaagaacctcaatcttctcctcaagaatctccttcttctcctgaatctgaatctcctccttctgaacctcctccttctgaagaagaagaacctcctgaacaagaagaacctcctgaatctgaagaagaacctgaagaagaagaagaagaagaagaagaacctgaagaagaagaagaagaaccttctgaagaatctcctgaatctgaatctgaacctcctcctccttcttctgaaccttctgaaccttctgaacctgaatctcctgaagaagaatcttctcctgaagaatctcaatctcctgaagaagaagaagaagaatctgaagaagaacctcaacctgaatcttctgaacctgaagaacctgaagaacaagaacaacaagaagaacaagaagaacctccttctcctcaacctcctgaagaacaacctcaacaacaagaacaagaacaatctgaaccttctgaacaacaagaacaaccttcttcttctcctgaatctgaagaagaatctgaacctgaagaacctgaacctgaacaagaatctcctcctgaatctgaagaagaatctgaacaacctcctgaatctccttcttctgaaccttcttctcctgaagaatctcaagaatcttcttctcctgaatctcctgaatctccttctcctcctgaatcttctcaacctgaagaagaacctcaacaagaacctgaaccttcttctcctcaacctcaagaacaacctgaagaagaagaatctcctcctccttcttctcctgaacaacctgaagaacctgaagaagaatcttcttctcaatcttctcaagaagaacaaccttctgaagaagaatctgaagaagaagaatctcaagaagaaccttctgaatcttctgaagaacctgaagaagaagaagaagaacctcctgaatctcaatctgaagaacaatctcaagaagaacaacctgaatctcctcaagaagaagaacaatctgaatctcctcctcaacctcctgaagaacctgaagaacaatcttctcaagaagaatctgaagaagaacaaccttctgaacaatcttctgaagaaccttcttctgaatctgaagaatctgaacctcaagaatctgaagaagaagaacctccttctgaacctgaatctgaacaacaatctgaagaacctcctcaatctcaagaagaatctcctcaaccttctccttctgaacctgaagaagaagaacaaccttctgaagaagaaccttctcaagaacaagaacctgaagaagaagaagaagaagaatcttctgaacctcctgaagaagaagaacctcaagaagaacctgaagaacctcctgaagaagaagaagaagaagaacaatctgaagaagaagaagaacctgaagaaccttctgaacaagaagaagaacctcctgaagaacctgaagaatctgaatctgaatctccttctcctgaaccttcttcttctgaacaatcttctccttctgaacaagaacaatcttctgaagaatctcaacctgaacctgaacctgaagaacaatctgaagaatcttctcaacctcctgaacctgaacctcctcctcctcctgaatctgaatcttcttcttctgaatctgaatctgaacaatctgaatctcaagaagaacctgaaccttctgaagaaccttctgaacaatcttctgaatctgaagaacctgaatctgaagaagaagaagaatctcctgaagaacctgaacaagaacaaccttctgaacctgaagaacctgaacctgaatctgaacaagaagaagaatctgaatctcctcctcctcctccttctgaagaatctcctcctcaatcttctgaaccttctcctgaagaacaacctcaagaatctgaacctgaacctgaaccttcttctcctcctgaacctcctcctgaagaagaatcttctgaacctgaatctgaagaagaatctgaatcttctgaacaagaacctgaagaacctcctgaatctgaatctgaa

SEQ ID NO: 21 - DNA, 3000 bp, Artificial Sequence
  Sequence was produced using the reverse translation tool located at www.vivo.colostate.edu/molkit/rtranslate/index.htm l.
gaagaagaagaatcttctcctcctgaagaagaagaatcttctcctgaacctgaagaacctgaacctgaaccttctcctcctcaagaagaagaagaagaaccttctcctcaagaacaacaacctcaacaacaagaatcttctcaagaagaagaacaagaacctgaagaagaagaacaagaatcttcttctcctcaagaagaacctcctcaacctgaagaagaacctgaacctgaagaagaagaagaatcttcttctgaagaagaagaacctgaagaacaagaacaacctgaacctgaagaagaaccttctcctgaatcttctgaatctgaatcttcttcttctgaagaagaagaagaacaaccttctcaacctgaatcttctccttctgaagaagaacaacctcaagaacctgaagaacctgaacctgaagaagaatctccttctcctcctgaagaacaagaagaagaatctgaatctgaagaagaacaagaacaatctgaacctgaagaatctgaagaagaagaagaaccttcttctcctcaatctgaacaagaagaacctcaagaacctgaacctgaagaacaagaagaagaacctcctgaagaagaagaacaagaacctcctgaatctgaatctcctgaagaacaagaagaagaacaacctccttctcctgaagaagaatctgaagaagaagaagaacctgaagaagaagaagaacaagaagaatctgaagaagaagaatctcaatctccttctgaagaacctgaacctgaagaatcttcttctcctgaatctgaagaacctcctgaagaagaatcttctgaagaatcttctgaagaatctcaagaagaatctccttctcctgaagaagaagaagaatcttctgaatctgaacaacctcctgaatctccttctgaatctcaagaatctccttctcaatctgaagaagaatctcaagaagaacctcctgaagaagaatcttctcctgaagaagaacctcctccttctccttctgaatctgaacctcctgaagaagaagaagaaccttctgaatctgaagaagaagaacctcctcctgaagaagaagaatcttcttctgaagaacaagaatctgaagaacctgaatctgaagaagaatctcctgaagaacaatctgaagaagaagaagaatctcaagaatcttctcctgaacctcctgaagaatctccttctgaacaacctgaaccttctcctcctgaacctgaatctgaatcttctgaacctgaagaagaagaagaagaagaagaagaacctccttcttctgaagaagaagaatctgaagaacctgaacaacctgaagaagaacaagaagaacctcaagaagaagaagaatctccttctgaagaatctcctgaagaacctgaagaatctgaacctgaagaagaatctgaagaagaagaacctgaacaacaacctgaagaagaacctcctgaagaagaagaacaagaatcttctgaaccttcttctcctccttctgaagaacaatctgaagaacctgaagaacaagaagaacctcctgaaccttctcaacctgaacctcaacaagaatctgaatcttcttctccttctgaatctcaacctgaatctcaagaatctgaagaagaagaagaagaagaagaatctgaagaagaatctgaaccttctcaagaacctgaagaacaacaacctgaagaagaagaagaagaagaagaagaacctgaagaagaagaagaacaatctgaacctgaagaatcttctgaacaacaagaacctcctcaatcttctcaacctcaagaagaatctgaacaagaacaagaagaacctcaatctcctgaagaagaatctcctcctcctgaagaagaagaacctcaagaagaacctcctgaacctgaagaagaagaaccttctgaacaacctccttcttctcctcctgaagaacaatctgaacaacctgaacaatctgaacctcaatctgaatctccttctcaacctgaatcttctgaacaacctgaagaacaacctgaacctccttctcctcaatcttctgaagaatctgaagaacctgaagaagaagaacaatctgaagaaccttctccttctcaatctgaatcttcttcttctcctgaagaatctgaacctcctgaagaagaagaagaagaagaagaacctgaagaacctgaacaagaagaagaacaatctgaacctcaagaacaagaaccttctgaagaatcttctgaacctgaagaagaatcttctccttcttctcaatcttctgaacaatcttcttctgaagaagaatctgaatctgaacaatcttctcctcctcctgaagaagaatctcctgaagaagaagaacctgaagaagaagaacctgaagaatctcctgaagaagaatctgaagaatctcctgaatctgaagaatctgaagaatcttctgaagaacaagaagaatcttctcctgaagaagaaccttctgaacaagaagaacctcctgaacaagaacctgaatctcctcctgaacaagaagaagaagaagaacaatctgaacctcaagaagaagaacctcctgaatcttctgaacctgaagaagaatctcctcctgaagaacctcaatctgaagaagaagaagaagaacctcaacctgaatctgaatctgaacctgaagaaccttctcctgaacctgaatctgaagaatctgaagaagaacctgaatctgaatcttcttctcctcctgaatcttcttctgaagaagaagaagaagaacctgaagaacaatctgaagaagaagaagaatctcaagaagaagaagaacaagaagaagaaccttctcaagaagaagaagaacctgaagaacaacaacctccttctgaagaagaagaacaacctgaacaatctgaagaacctgaaccttctgaaccttctgaagaagaacctgaacctgaagaatctcctcctgaatctcaacctccttctgaagaacct

SEQ ID NO: 22 - DNA, 3000 bp, Artificial Sequence
  Sequence was produced using the reverse translation tool located at www.vivo.colostate.edu/molkit/rtranslate/index.htm l.
ggtcaacaaggttcttctcctccttctccttctcaaggtggtcaacctccttcttctcaaccttctcaacaatcttcttcttctcctcctccttctcctcctccttcttctcctccttctcaacctccttctcctccttcttctggttctggttcttcttctccttctcaaggttctcctccttctcctccttctcaaggtcctcctcaacctcctcaatctcctggttctcaaggtcctcctcctcctcctggtcctggttctggtcctcctccttcttcttctcctcaaccttctcaacctcctccttctcaaccttctcaacaatctcctcaaccttctcctggtcctggttctccttctcaacaaccttcttctggttctcaacaatctcctggtcaaggtcctcaacctcaaggtccttctggttctcctcaaggtcaaggttctcctggttcttcttctggtcctcaaccttcttctcaaggttctcctcctggtcctcctcctggtccttctccttctggtggtcctcaatcttctcctggttctcctccttctcctcaaggttctcaacctcaatctcctggtccttcttctccttcttcttctcctcaacctccttctggtcctccttcttctggtggtcaatcttctcaaggtcaatctccttctcaaggtcctcctcctggttctcctcaacctcctggtggttctggtccttctccttcttcttctcctcctccttctcctcctcctcctcaatcttcttcttctggttctcaacaatcttcttcttcttctggttctcctccttcttcttctcaaggtcctcctcaatcttcttctcaacctcaatctcaatcttctccttctcaacctccttctggttctcctggttcttcttcttctccttctccttctccttctggtccttctggttctccttctggtcctccttcttctccttctggttctcctcctcctggtggtcctcctcaatctggtggtcctggtccttcttctggtcaacaacctcctggtcctcaacctggttctcctcctggtcaacctcaacctggttcttcttctcaaggtcctcaacaaggtcctcctcctggttctcctcaaggtccttctcaacctggtcctcaatctcctccttcttctggtggttcttcttctcaacctcaatctccttcttctggtcctggtcaaccttctccttctcctcctggttctcctggtggtcctggtcaacctccttctcaaccttctccttcttcttcttcttctcaatctggtcaatcttctcaaccttctggtcctccttctggtcaatctcaacctggtcaacctcctcaaccttctcctccttctcctcctcctccttctcctccttctcaatctggttctggttctcctggtcctccttctggtcctcaaccttcttctcaaccttctccttctcaacctggtcaaggtccttcttcttctcctcctggtcaatctggtccttcttctccttcttcttctcaacctcctccttctcaatctcctcctcaatctggtcaatctccttcttcttctcctcctcaatcttctccttcttctggtcaacaaccttctcctggtcctccttcttcttcttctcctcaaccttcttcttctcaaggttctcctcctcctcaacctcaaggtcaatctcctccttctcaacaaccttctcaacctggtggttcttctcaaccttcttctcctcctcctcctggtcctcaaggtcctcaacctccttctcctcaacctccttctggtcctggttctcaacctcaaggtggttctccttcttctcaaggtggtcaaccttcttcttctcctcctcaatcttcttctggtccttctggtcctggttcttctccttctcaatctccttctggtcaaggtccttcttctcaaccttctccttctggttctggtcaacctcaaggtcctccttctccttctggtcaacctccttctcctccttctggttctccttctcctcctcaacctggttctcctggtcaacctcaaccttctcctccttctcaatctcctggtggtcctggtggtcctcaaggtcctccttcttctcctggttcttctggttcttctggttcttctcaacctcctcctcctccttctcaacaatcttcttctggtcaatctcctcaacctcaaggtcaaggtcaacaacctggttctcctggtcaatctggtcaacaatctcaatctcctggtggtccttctcctcaacaacctcctcctcctcctcctcctcctcctggttcttctcctcaatcttctcctcaaccttctccttctcaatctcaacctcaatctggttctcaatcttctcaacaacaatctcaatcttcttcttctccttctcctcaatctcaaggtggtcctcaatcttctggttcttctccttcttctggtcctcaatctccttctcctggtggtcctcctccttctcaatcttcttctggtcaaccttctcctccttctcctcctggtccttctggttcttcttcttcttcttctggttctggttctggtcctcaaccttctcctcctcctcaatctccttctcaacaatctggttcttctcaatcttctccttctcaatctcaacctcaacctcctcctcctggttctggtcaacctcctccttctggtggtcctcaacaacctccttctcctcaacaaggttctcaatcttcttctcaacctcctcctcctcaatcttcttcttctggtggtcctggtcaatcttctggttctcctggtccttctcctcctcaacaatctggtggttctcctcctccttctggtggtggttctggtcctggttctcctccttctggtcaaggttctccttctcaatcttctggtccttctggtggtcctggtggttctcctcctcctccttcttctccttctccttctcaatcttct

SEQ ID NO: 23 - protein, 100 aa, Artificial Sequence
  The sequence was produced using the random sequence generator tool located at the Swiss-Prot website http//au.expasy.org/tools/randseq.html.
PSKSPSPKPPQPSKPPQSKKPQSQSPPPQSSPKSPPKPPQSKQQPSSPSPQQPSKKSSSSQSQPSQKSSPKSSKPPPSQKPPKPKPKPPPKSPQSKPQQK

SEQ ID NO: 24 - protein, 30 aa, Artificial Sequence
  The sequence was produced using the random sequence generator tool located at the Swiss-Prot website http//au.expasy.org/tools/randseq.html.
KSPPKPPQSKQQPSSPSPQQPSKKSSSSQS

SEQ ID NO: 25 - protein, 100 aa, Artificial Sequence
  The sequence was produced using the random sequence generator tool located at the Swiss-Prot website http//au.expasy.org/tools/randseq.html.
PSESPSPEPPQPSEPPQSEEPQSQSPPPQSSPESPPEPPQSEQQPSSPSPQQPSEESSSSQSQPSQESSPESSEPPPSQEPPEPEPEPPPESPQSEPQQE

SEQ ID NO: 26 - protein, 30 aa, Artificial Sequence
  The sequence was produced using the random sequence generator tool located at the Swiss-Prot website http//au.expasy.org/tools/randseq.html.
ESPPEPPQSEQQPSSPSPQQPSEESSSSQS

SEQ ID NO: 27 - protein, 100 aa, Artificial Sequence
  The sequence was produced using the random sequence generator tool located at the Swiss-Prot website http//au.expasy.org/tools/randseq.html.
PSGSPSPGPPQPSGPPQSGGPQSQSPPPQSSPGSPPGPPQSGQQPSSPSPQQPSGGSSSSQSQPSQGSSPGSSGPPPSQGPPGPGPGPPPGSPQSGPQQG

SEQ ID NO: 28 - protein, 30 aa, Artificial Sequence
  The sequence was produced using the random sequence generator tool located at the Swiss-Prot website http//au.expasy.org/tools/randseq.html.
GSPPGPPQSGQQPSSPSPQQPSGGSSSSQS

SEQ ID NO: 29 - DNA, 300 bp, Artificial Sequence
  Sequence was produced using the reverse translation tool located at www.vivo.colostate.edu/molkit/rtranslate/index.htm l
ccttctaaatctccttctcctaaacctcctcaaccttctaaacctcctcaatctaaaaaacctcaatctcaatctcctcctcctcaatcttctcctaaatctcctcctaaacctcctcaatctaaacaacaaccttcttctccttctcctcaacaaccttctaaaaaatcttcttcttctcaatctcaaccttctcaaaaatcttctcctaaatcttctaaacctcctccttctcaaaaacctcctaaacctaaacctaaacctcctcctaaatctcctcaatctaaacctcaacaaaaa

SEQ ID NO: 30 - DNA, 90 bp, Artificial Sequence
  Sequence was produced using the reverse translation tool located at www.vivo.colostate.edu/molkit/rtranslate/index.htm l
aaatctcctcctaaacctcctcaatctaaacaacaaccttcttctccttctcctcaacaaccttctaaaaaatcttcttcttctcaatct

SEQ ID NO: 31 - DNA, 300 bp, Artificial Sequence
  Sequence was produced using the reverse translation tool located at www.vivo.colostate.edu/molkit/rtranslate/index.htm l
ccttctgaatctccttctcctgaacctcctcaaccttctgaacctcctcaatctgaagaacctcaatctcaatctcctcctcctcaatcttctcctgaatctcctcctgaacctcctcaatctgaacaacaaccttcttctccttctcctcaacaaccttctgaagaatcttcttcttctcaatctcaaccttctcaagaatcttctcctgaatcttctgaacctcctccttctcaagaacctcctgaacctgaacctgaacctcctcctgaatctcctcaatctgaacctcaacaagaa

SEQ ID NO: 32 - DNA, 90 bp, Artificial Sequence
  Sequence was produced using the reverse translation tool located at www.vivo.colostate.edu/molkit/rtranslate/index.htm l
gaatctcctcctgaacctcctcaatctgaacaacaaccttcttctccttctcctcaacaaccttctgaagaatcttcttcttctcaatct

SEQ ID NO: 33 - protein, 300 aa, Artificial Sequence
  Sequence was produced using the reverse translation tool located at www.vivo.colostate.edu/molkit/rtranslate/index.htm l
CCTTCTGGTTCTCCTTCTCCTGGTCCTCCTCAACCTTCTGGTCCTCCTCAATCTGGTGGTCCTCAATCTCAATCTCCTCCTCCTCAATCTTCTCCTGGTTCTCCTCCTGGTCCTCCTCAATCTGGTCAACAACCTTCTTCTCCTTCTCCTCAACAACCTTCTGGTGGTTCTTCTTCTTCTCAATCTCAACCTTCTCAAGGTTCTTCTCCTGGTTCTTCTGGTCCTCCTCCTTCTCAAGGTCCTCCTGGTCCTGGTCCTGGTCCTCCTCCTGGTTCTCCTCAATCTGGTCCTCAACAAGGT

SEQ ID NO: 34 - DNA, 90 bp, Artificial Sequence
  Sequence was produced using the reverse translation tool located at www.vivo.colostate.edu/molkit/rtranslate/index.htm l
ggttctcctcctggtcctcctcaatctggtcaacaaccttcttctccttctcctcaacaaccttctggtggttcttcttcttctcaatct

SEQ ID NO: 35 - protein, 100 aa, Artificial Sequence
  The sequence was produced using the random sequence generator tool located at the Swiss-Prot website http//au.expasy.org/tools/randseq.html.
PSXSPSPXPPQPSXPPQSXXPQSQSPPPQSSPXSPPXPPQSXQQPSSPSPQQPSXXSSSSQSQPSQXSSPXSSXPPPSQXPPXPXPXPPPXSPQSXPQQX

(19) United States
(12) Patent Application Publication
(10) Pub. No.: US 2009/0137004 A1
(43) Pub. Date: May 28, 2009

(54) ARTIFICIAL ENTROPIC BRISTLE DOMAIN SEQUENCES AND THEIR USE IN RECOMBINANT PROTEIN PRODUCTION

(75) Inventors: Vladimir N. Uversky, Carmel, IN (US); A. Keith Dunker, Indianapolis, IN (US)

Correspondence Address: SEED INTELLECTUAL PROPERTY LAW GROUP PLLC, 701 FIFTH AVE, SUITE 5400, SEATTLE, WA, 98104, US

(73) Assignee: MOLECULAR KINETICS INCORPORATED, Indianapolis, IN (US)

(21) Appl. No.: 12/272,558

(22) Filed: Nov. 17, 2008

Related U.S. Application Data
(60) Provisional application No. 60/988,319, filed on Nov. 15, 2007.

(51) Int. Cl. C12P 21/04; C07K 14/00; C12N 15/11; C12N 15/00; C12N 1/21

(52) U.S. Cl. 435/69.7; 530/350; 530/324; 536/23.4; 435/320.1; 435/252.33

(57) ABSTRACT

Compositions and methods for recombinant protein production and, more particularly, fusion polypeptides, polynucleotides encoding fusion polypeptides, expression vectors, kits, and related methods for recombinant protein production.

### CROSS-REFERENCE TO RELATED APPLICATION

This application claims the benefit under 35 U.S.C. § 119(e) of U.S. Provisional Patent Application No. 60/988,319, filed Nov. 15, 2007; where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 670098_{—}406_SEQUENCE_LISTING.txt. The text file is 142 KB, was created on Nov. 17, 2008, and is being submitted electronically via EFS-Web, concurrent with the filing of the specification.

### FIELD OF THE INVENTION

The present invention relates generally to compositions and methods for improved recombinant protein production and, more particularly, to fusion polypeptides, polynucleotides encoding fusion polypeptides, expression vectors, kits, and related methods for recombinant protein production.

### DETAILED DESCRIPTION OF THE RELATED ART

A large percentage of the proteins identified via the different genome sequencing effort have been difficult to express and/or purify as recombinant proteins using standard methods. For example, a trial study using Methanobacterium thermoautotrophicum as a model system identified a number of problems associated with high throughput structure determination (Christendat et al. (2000) Prog. Biophys. Mol. Biol. 73(5): 339-345; Christendat et al. (2000) Nat Struct Biol 7(10): 903-909). The complete list of genome-encoded proteins was filtered to remove proteins with predicted transmembrane regions or homologues to known structures. When these filtered proteins were taken through the cloning, expression, and structural determination steps of a high throughput process, only about 50% of the selected proteins could be purified in a state suitable for structural studies, with roughly 45% of large expressed proteins and 30% of small expressed proteins failing due to insolubility. The study concluded that considerable effort must be invested in improving the attrition rate due to proteins with poor expression levels and unfavorable biophysical properties. (Christendat et al. (2000) Prog. Biophys. Mol. Biol. 73(5): 339-345; Christendat et al. (2000) Nat Struct Biol 7(10): 903-909).

Similar results have been observed for other prokaryotic proteomes. One study reported the successful cloning and attempted expression of 1376 (73%) of the predicted 1877 genes of the Thermotoga maritima proteome. However, crystallization conditions were able to be determined for only 432 proteins (23%). A significant component of the decrease between the cloned and crystallized success levels was due to poor protein solubility and stability (Kuhn et al. (2002) Proteins 49(1): 142-5).

Similarly low success rates have been reported for eukaryotic proteomes. A study of a sample set of human proteins, for example, reported that the failure rate using high-throughput methods for three classes of proteins based on cellular location was 50% for soluble proteins, 70% for extracellular proteins, and more than 80% for membrane proteins (Braun et al. (2002) Proc Natl Acad Sci USA 99(5): 2654-9).

Interactions between individual recombinant proteins are responsible for a significant number of the previously mentioned failures. In a high-throughput structural determination study, Christendat and colleagues found that 24 of 32 proteins that were classified by nuclear magnetic resonance as aggregated displayed circular dichroism spectra consistent with stable folded proteins, suggesting that these proteins were folded properly but aggregated due to surface interactions (Christendat et al. (2000) Prog. Biophys. Mol. Biol. 73(5): 339-345). One possible explanation for this is that these proteins function in vivo as part of multimeric units but when they are recombinantly expressed, dimerization domains are exposed that mediate protein-protein interactions.

Prior methods used to increase recombinant protein stability include production in E. coli strains that are deficient in proteases (Gottesman and Zipser (1978) J Bacteriol 133(2): 844-51) and production of fusions of bacterial protein fragments to a recombinant polypeptide/protein of interest (Itakura et al., Science, 1977. 198:1056-63; Shen, Proc Natl Acad Sci USA, 1984. 81:4627-31). It has also been attempted to stabilize foreign proteins in E. coli. In addition, fusing a leader sequence to a recombinant protein may cause a gene product to accumulate in the periplasm or be excreted, which may result in increased recovery of properly folded soluble protein (Nilsson et al., EMBO J, 1985. 4:1075-80; Abrahmsen et al., Nucleic Acids Res, 1986. 14:7487-500). These strategies have advantages for some proteins but they generally do not succeed when used, for example, with membrane proteins or proteins capable of strong protein-protein interactions.

Fusion polypeptides have also been used as an approach for improving the solubility and folding of recombinant polypeptides/proteins produced in E. coli (Zhan et al., Gene, 2001. 281:1-9). Some commonly used fusion partners which have been linked to heterologous protein sequences of interest include calmodulin-binding peptide (CBP) (Vaillancourt et al., Biotechniques, 1997. 22:451-3), glutathione-S-transferase (GST) (Smith, Methods Enzymol, 2000. 326:254-70), thioredoxin (TRX) (Martin Hammarström et al., Protein Science, 2002. 11:313-321), and maltose-binding protein (MBP) (Sachdev et al., Methods Enzymol, 2000. 326:312-21). Glutathione-S-transferase and maltose-binding protein have been found to increase the recombinant protein purification success rate when fused to a heterologous sequence in a controlled trial of 32 human test proteins (Braun et al., Proc Natl Acad Sci USA, 2002. 99:2654-9). Further, maltose-binding protein domain fusions have been shown to increase the solubility of recombinant proteins (Kapust et al., Protein Sci, 1999. 8:1668-74; Braun et al., Proc Natl Acad Sci USA, 2002. 99:2654-9; Martin Hammarström et al., Protein Science, 2002. 11:313-321). Maltose-binding protein may further benefit recombinant protein solubility and folding in that it may have chaperone-like properties that assist in folding of the fusion partner (Richarme et al., J Biol Chem, 1997. 272:15607-12; Bach et al., J Mol Biol, 2001. 312:79-93. However, these fusion approaches used to date have not been amendable to all classes of proteins, and have thus met with only limited success.

Entropic bristles have been used in a variety of polymers to reduce aggregation of small particles such as latex particles in paints and to stabilize a wide variety of other colloidal products (Hoh, Proteins, 1998. 32:223-228). Entropic bristles generally comprise amino acid residues that do not have a tendency to form secondary structure and in the process of random motion about their attachment points sweep out a significant region in space and entropically exclude other molecules by their random motion (Hoh, Proteins, 1998. 32:223-228). Entropic bristles are singular elements, comprising highly flexible, non-aggregating polymer chains, of which entropic brushes are assembled. In polymer chemistry, entropic bristles have been affixed to the surfaces of particles (e.g. latex beads), thereby forming entropic brushes which, in turn, prevent particle aggregation (Stabilization by attached polymer: steric stabilization, in Polymeric stabilization of colloidal dispersions, D. H. Napper, Editor. 1983, Academic Press: London. p. 18-30). EBDs can exclude large molecules but do not exclude small molecules such as water, salts, metal ions, or cofactors (Hoh, Proteins, 1998. 32:223-228).

EBDs can also function as steric stabilizers and operate through steric hindrance stabilization (Stabilization by attached polymer: steric stabilization, in Polymeric stabilization of colloidal dispersions, D. H. Napper, Editor. 1983, Academic Press: London. p. 18-30). Naper described characteristics that contribute to steric stabilization functions, including (1) they have an amphipathic sequence; (2) they are attached to the colloidal particle by one end rather than being totally adsorbed; (3) they are soluble in the medium used; (4) they are mutually repulsive; (5) they are thermodynamically stable; and (6) they exhibit stabilizing ability in proportion to their length. Steric stabilizers intended to function in aqueous media extend from the surface of colloidal molecules thus transforming their surfaces from hydrophobic to hydrophilic. The fact that sterically stabilized particles are thermodynamically stable leads them to spontaneously re-disperse when dried residue is reintroduced to solvent. Entropic bristles can adopt random-walk configurations in solution (Milner, Science, 1991. 251:905-914). These chains extend from an attachment point because of their affinity for the solvent. This affinity is due in part to the highly charged nature of the entropic bristle sequence.

While naturally-occurring EBDs possess features desirable for use in improving the solubility, folding, etc., of recombinant proteins, prior attempts at using EBD sequences in fusion with heterologous protein sequences have met with limited success, due in part to cellular toxicity associated with the naturally occurring EBDs. Accordingly, there remains a need for new compositions and methods for improving the properties and characteristics of recombinant proteins, e.g., improving solubility, stability, yield and/or folding of recombinant proteins. The present invention addresses these needs and offers other related advantages by providing non-naturally occurring EBD sequences as fusion partners for use in recombinant protein production techniques, as described herein.

### SUMMARY OF THE INVENTION

According to a general aspect of the present invention, there are provided isolated fusion polypeptides comprising at least one artificial, non-naturally occurring entropic bristle domain (EBD) sequence and at least one heterologous polypeptide sequence of interest. The fusion polypeptides comprising artificial EBD sequences as described herein offer a number of advantages over prior fusion polypeptides and methods relating thereto. For example, the fusion polypeptides of the invention offer increased solubility relative to the heterologous polypeptide sequence, reduced aggregation relative to the heterologous polypeptide sequence and/or improved folding relative to the heterologous polypeptide sequence.

In one illustrative embodiment, the invention provides fusion polypeptides comprising at least one non-naturally occurring entropic bristle domain (EBD) polypeptide sequence and at least one heterologous polypeptide sequence to be expressed, wherein the EBD polypeptide sequence is about 10-1000 amino acid residues in length, and wherein at least 75% of the residues of the EBD polypeptide sequence are selected from G, D, M, K, R, S, Q, P, and E. In other embodiments, at least 80, 85, 90 or 95% of the residues of the EBD polypeptide sequence are selected from G, D, M, K, R, S, Q, P, and E.

In another illustrative embodiment, the EBD polypeptide sequence is positively charged and the amino acid residues which make up the EBD polypeptide comprise disorder-promoting amino acid residues selected from P, Q, S and K. In a more specific embodiment, the disorder-promoting amino acid residues P, Q, S and K are present in about the following amino acid ratios: K:P:Q:S=1:2:1:2, K:P:Q:S=2:2:1:2, K:P:Q:S=3:2:1:2, K:P:Q:S=4:2:1:2, or K:P:Q:S=5:2:1:2. In a more specific embodiment, the EDB polypeptide sequence comprises a sequence set forth in SEQ ID NO: 1, SEQ ID NO: 2, SEQ ID NO: 3, SEQ ID NO: 4, SEQ ID NO: 5, SEQ ID NO: 23, or SEQ ID NO: 24, or a fragment thereof, or a sequence having at least 90% identity thereto.

In another illustrative embodiment, the EBD polypeptide sequence is negatively charged and the amino acid residues are disorder-promoting amino acid residues selected from P, Q, S and E. In a more specific embodiment, the disorder-promoting amino acid residues P, Q, S and E are present in about the following amino acid ratios: E:P:Q:S=1:2:1:2, E:P:Q:S=2:2:1:2, E:P:Q:S=3:2:1:2, E:P:Q:S=4:2:1:2, or E:P:Q:S=5:2:1:2. In a more specific embodiment, the EDB polypeptide comprises the sequence set forth in SEQ ID NO: 6, SEQ ID NO: 7, SEQ ID NO: 8, SEQ ID NO: 9, SEQ ID NO: 10, SEQ ID NO: 25, or SEQ ID NO: 26, or a fragment thereof, or a sequence having at least 90% identity thereto.

In yet another illustrative embodiment, the EBD polypeptide sequence is neutral and the disorder-promoting residues are selected from P, Q, S and G. In a more particular embodiment, the amino acid residues P, Q, S and G are present in about the amino acid ratio of G:P:Q:S=1:2:1:2. In a more particular embodiment, the EDB polypeptide comprises the sequence set forth in SEQ ID NO: 11, SEQ ID NO: 27, or SEQ ID NO: 28, or a fragment thereof, or a sequence having at least 90% identity thereto.

In another illustrative embodiment, the EBD polypeptide sequence is positively charged and the amino acid residues are disorder-promoting amino acid residues selected from P, Q, S and R. In a more specific embodiment, the amino acid residues R, P, Q and S are present in about the following amino acid ratios: R:P:Q:S=1:2:1:2, R:P:Q:S=2:2:1:2, R:P:Q:S=3:2:1:2, R:P:Q:S=4:2:1:2, or R:P:Q:S=5:2:1:2.

In another illustrative embodiment, the EBD polypeptide sequence is negatively charged and the amino acid residues are disorder-promoting amino acid residues are selected from P, Q, S and D. In a more particular embodiment, the amino acid residues D, P, Q and S are present in about the following amino acid ratios: D:P:Q:S=1:2:1:2, D:P:Q:S=2:2:1:2, D:P:Q:S=3:2:1:2, D:P:Q:S=4:2:1:2, or D:P:Q:S=5:2:1:2.

A fusion polypeptide of the invention, comprising an EBD sequence and a heterologous polypeptide sequence, exhibits improved solubility relative to the corresponding heterologous polypeptide in the absence of the EBD sequence. In a related embodiment, the fusion polypeptide has at least 5% increased solubility relative to the heterologous polypeptide sequence, at least 25% increased solubility relative to the heterologous polypeptide sequence, or at least 50% increased solubility relative to the heterologous polypeptide sequence.

In another embodiment, a fusion polypeptide of the invention exhibits reduced aggregation relative to the level of aggregation of the heterologous polypeptide sequence in the absence of the EBD sequence. For example, a fusion polypeptide of the invention generally exhibits at least 10% reduced aggregation relative to the heterologous polypeptide sequence or at least 25% reduced aggregation relative to the heterologous polypeptide sequence.

In another embodiment, a fusion polypeptide of the invention exhibits improved self-folding relative to the heterologous polypeptide sequence in the absence of the EBD sequence.

In another embodiment of the present invention, an EBD sequence employed in a fusion polypeptide comprises an amino acid sequence that maintains a substantially random coil conformation.

In another embodiment, the EBD sequence of a fusion polypeptide of the invention comprises an amino acid sequence that is substantially mutually repulsive.

In another embodiment, the EBD sequence of a fusion polypeptide of the invention comprises an amino acid sequence that remains in substantially constant motion.

In another embodiment of the present invention, the EBD sequence of a fusion polypeptide of the invention is a random sequence of disorder-promoting amino acid residues.

The EBD sequence of a fusion polypeptide of the invention generally comprises between about 5 to 1000 amino acid residues, 5 to 500 amino acid residues, 5 to 400 amino acid residues, 5 to 300 amino acid residues, 5 to 200 amino acid residues, 5 to 100 amino acid residues, 5 to 80 amino acid residues, 5 to 60 amino acid residues, 5 to 40 amino acid residues, 5 to 30 amino acid residues, 5 to 20 amino acid residues, 10 to 30 amino acid residues, 15 to 25 amino acid residues, 10 to 90 amino acid residues, 20 to 80 amino acid residues, 20 to 40 amino acid residues, 30 to 70 amino acid residues, or 40 to 60 amino acid residues.

In a related embodiment, the disorder-promoting EBD sequence comprises no more than about 20 amino acid residues, 30 amino acid residues, 40 amino acid residues, 50 amino acid residues, 100 amino acid residues, 200 amino acid residues, 300 amino acid residues, 400 amino acid residues, 500 amino acid residues, or 1000 amino acid residues.

In yet another related embodiment, the EBD sequence of a fusion polypeptide of the invention comprises at least 2-100 repeats of an EBD sequence set forth above or described herein, or a combination thereof.

In another embodiment, the EBD sequence of a fusion polypeptide of the invention comprises a combination of any one or more of fragments derived from disorder-promoting EBD sequences that are positively charged, negatively charges, or neutral as set here herein.

In another embodiment, an EBD sequence of a fusion polypeptide of the invention is cleavable, e.g., can be removed and/or separated from the heterologous polypeptide sequence after recombinant expression by, for example, enzymatic or chemical cleavage methods.

In another embodiment, an EBD sequence of a fusion polypeptide of the invention is covalently linked at the N-terminus of the heterologous polypeptide sequence of interest. In another embodiment, an EBD sequence of a fusion polypeptide of the invention is covalently linked at the C-terminus of the heterologous polypeptide sequence of interest. In yet another embodiment, an EBD sequence of a fusion polypeptide of the invention is covalently linked at the N- and C-termini of the heterologous polypeptide sequence of interest.

In another embodiment of the invention, the charge of an EBD sequence of a fusion polypeptide of the invention is modulated by, for example, enzymatic and/or chemical methods, in order to modulate the activity of the EBD sequence. In a particular embodiment, the charge of the EBD sequence is modulated by phosphorylation.

According to another aspect of the invention, an isolated polynucleotide is provided, wherein the polynucleotide encodes a fusion polypeptide as described herein or an artificial EBD sequence as described herein.

According to yet another aspect of the invention, there is provided an expression vector comprising an isolated polynucleotide encoding a fusion polypeptide as described herein or an artificial EBD sequence as described herein. In a related embodiment, an expression vector is provided comprising a polynucleotide encoding an EBD sequence and further comprising a cloning site for insertion of a polynucleotide encoding a heterologous polypeptide of interest.

According to yet another aspect of the invention, there is provided a host cell comprising an expression vector as described herein.

According to yet another aspect of the invention, there is provided a kit comprising an isolated polynucleotide as described herein, an isolated polypeptide as described herein and/or an isolated host cell as described herein.

Yet another aspect of the invention provides a method for producing a recombinant protein comprising the steps of: introducing into a host cell an expression vector comprising a polynucleotide sequence encoding a fusion polypeptide, the fusion polypeptide comprising at least one EBD sequence and at least one polypeptide sequence of interest; and expressing the fusion polypeptide in the host cell. In another embodiment, the method further comprises the step of isolating the fusion polypeptide from the host cell. In another related embodiment, the method further comprises the step of removing the EBD sequence from the fusion polypeptide before or after isolating the fusion polypeptide from the host cell.

These and other aspects of the present invention will become apparent upon reference to the following detailed description. All references disclosed herein and in the enclosed Application Data Sheet are hereby incorporated by reference in their entirety as if each was incorporated individually.

### BRIEF DESCRIPTION OF THE DRAWINGS

FIG. 1. Amino acid composition, relative to the set of globular proteins Globular-3D, of intrinsically disordered regions 10 residues or longer from the DisProt database. Slanted hash marks indicate DisProt 1.0 (152 proteins), while white indicates DisProt 3.4 (460 proteins). Amino acid compositions were calculated per disordered regions and then averaged. The arrangement of the amino acids is by peak height for the DisProt 3.4 release. Confidence intervals were estimated using per-protein bootstrapping with 10,000 iterations.

FIGS. 2A and 2B. Amino acid sequence of the randomly generated artificial EB containing the chosen residues in the following proportion: X:P:Q:S=1:2:1:2 (SEQ ID NO:35); X=K, E or G (2A) and sequences of positive, negative and neutral bristles, indicated as EB₊ (SEQ ID NO:24), EB₋ (SEQ ID NO:26) and EB₀ (SEQ ID NO:28) (2B), respectively. The actual X:P:Q:S ratios for these sequences was 5:8:6:11, numbers that are close to the 1:2:1:2 used to generate the sequences.

FIG. 3. Ligation of two DNA sequences via PCR. I, amplification of DNA1 and DNA2 sequences using reversed DNA1 overlapping primer P2 and DNA2 forward overlapping primer P3. II, Products of the PCR1 bearing overlapping fragments. III, PCR2 annealing step. IV. Final product composed of DNA1+DNA2.

FIGS. 4A and 4B. Expression and solubility of ten C. thermocellum proteins with N-terminal entropic bristles induced at 37° C. (4A), or MBP-fusions induced at 37° C. and 30° C. (4B). Abbreviations: T, total protein, S, soluble protein, U, uninduced cells. IDs of solubilized proteins and the corresponding EBDs are shown initalics.

### BRIEF DESCRIPTION OF THE SEQUENCE IDENTIFIERS

SEQ ID NO: 1 is the amino acid sequence of a positively charged EBD domain, EBD(+), which is a random sequence containing disorder-promoting residues P, Q, S and K in about the following amino acid ratios: K:P:Q:S=1:2:1:2. The sequence was produced using the random sequence generator tool located at the Swiss-Prot website: http://au.expasy.org/tools/randseq.html.

SEQ ID NO: 2 is the amino acid sequence of a positively charged EBD domain, EBD(++), which is a random sequence containing disorder-promoting residues P, Q, S and K in about the following amino acid ratios: K:P:Q:S=2:2:1:2. The sequence was produced using the random sequence generator tool located at the Swiss-Prot website: http://au.expasy.org/tools/randseq.html.

SEQ ID NO: 3 is the amino acid sequence of a positively charged EBD domain, EBD(+++), which is a random sequence containing disorder-promoting residues P, Q, S and K in about the following amino acid ratios: K:P:Q:S=3:2:1:2. The sequence was produced using the random sequence generator tool located at the Swiss-Prot website: http://au.expasy.org/tools/randseq.html.

SEQ ID NO: 4 is the amino acid sequence of a positively charged EBD domain, EBD(++++), which is a random sequence containing disorder-promoting residues P, Q, S and K in about the following amino acid ratios: K:P:Q:S=4:2:1:2. The sequence was produced using the random sequence generator tool located at the Swiss-Prot website: http://au.expasy.org/tools/randseq.html.

SEQ ID NO: 5 is the amino acid sequence of a positively charged EBD domain, EBD(+++++), which is a random sequence containing disorder-promoting residues P, Q, S and K in about the following amino acid ratios: K:P:Q:S=5:2:1:2. The sequence was produced using the random sequence generator tool located at the Swiss-Prot website: http://au.expasy.org/tools/randseq.html.

SEQ ID NO: 6 is the amino acid sequence of a negatively charged EBD domain, EBD(−), which is a random sequence containing disorder-promoting residues P, Q, S and E in about the following amino acid ratios: E:P:Q:S=1:2:1:2. The sequence was produced using the random sequence generator tool located at the Swiss-Prot website: http://au.expasy.org/tools/randseq.html.

SEQ ID NO: 7 is the amino acid sequence of a negatively charged EBD domain, EBD(−−), which is a random sequence containing disorder-promoting residues P, Q, S and E in about the following amino acid ratios: E:P:Q:S=2:2:1:2. The sequence was produced using the random sequence generator tool located at the Swiss-Prot website: http://au.expasy.org/tools/randseq.html.

SEQ ID NO: 8 is the amino acid sequence of a negatively charged EBD domain, EBD(−−−), which is a random sequence containing disorder-promoting residues P, Q, S and E in about the following amino acid ratios: E:P:Q:S=3:2:1:2. The sequence was produced using the random sequence generator tool located at the Swiss-Prot website: http://au.expasy.org/tools/randseq.html.

SEQ ID NO: 9 is the amino acid sequence of a negatively charged EBD domain, EBD(−−−−), which is a random sequence containing disorder-promoting residues P, Q, S and E in about the following amino acid ratios: E:P:Q:S=4:2:1:2. The sequence was produced using the random sequence generator tool located at the Swiss-Prot website: http://au.expasy.org/tools/randseq.html.

SEQ ID NO: 10 is the amino acid sequence of a negatively charged EBD domain, EBD(−−−−−), which is a random sequence containing disorder-promoting residues P, Q, S and E in about the following amino acid ratios: E:P:Q:S=5:2:1:2. The sequence was produced using the random sequence generator tool located at the Swiss-Prot website: http://au.expasy.org/tools/randseq.html.

SEQ ID NO: 11 is the amino acid sequence of a neutral EBD domain, EBD(0), which is a random sequence containing disorder-promoting residues P, Q, S and G in about the following amino acid ratios: G:P:Q:S=1:2:1:2. The sequence was produced using the random sequence generator tool located at the Swiss-Prot website: http://au.expasy.org/tools/randseq.html.

SEQ ID NO: 12 is a polynucleotide sequence encoding the amino acid sequence of SEQ ID NO: 1. Sequence was produced using the reverse translation tool located at: www.vivo.colostate.edu/molkit/rtranslate/index.html.

SEQ ID NO: 13 is a polynucleotide sequence encoding the amino acid sequence of SEQ ID NO: 2. Sequence was produced using the reverse translation tool located at: www.vivo.colostate.edu/molkit/rtranslate/index.html.

SEQ ID NO: 14 is a polynucleotide sequence encoding the amino acid sequence of SEQ ID NO: 3. Sequence was produced using the reverse translation tool located at: www.vivo.colostate.edu/molkit/rtranslate/index.html.

SEQ ID NO: 15 is a polynucleotide sequence encoding the amino acid sequence of SEQ ID NO: 4. Sequence was produced using the reverse translation tool located at: www.vivo.colostate.edu/molkit/rtranslate/index.html.

SEQ ID NO: 16 is a polynucleotide sequence encoding the amino acid sequence of SEQ ID NO: 5. Sequence was produced using the reverse translation tool located at: www.vivo.colostate.edu/molkit/rtranslate/index.html.

SEQ ID NO: 17 is a polynucleotide sequence encoding the amino acid sequence of SEQ ID NO: 6. Sequence was produced using the reverse translation tool located at: www.vivo.colostate.edu/molkit/rtranslate/index.html.

SEQ ID NO: 18 is a polynucleotide sequence encoding the amino acid sequence of SEQ ID NO: 7. Sequence was produced using the reverse translation tool located at: www.vivo.colostate.edu/molkit/rtranslate/index.html.

SEQ ID NO: 19 is a polynucleotide sequence encoding the amino acid sequence of SEQ ID NO: 8. Sequence was produced using the reverse translation tool located at: www.vivo.colostate.edu/molkit/rtranslate/index.html.

SEQ ID NO: 20 is a polynucleotide sequence encoding the amino acid sequence of SEQ ID NO: 9. Sequence was produced using the reverse translation tool located at: www.vivo.colostate.edu/molkit/rtranslate/index.html.

SEQ ID NO: 21 is a polynucleotide sequence encoding the amino acid sequence of SEQ ID NO: 10. Sequence was produced using the reverse translation tool located at: www.vivo.colostate.edu/molkit/rtranslate/index.html.

SEQ ID NO: 22 is a polynucleotide sequence encoding the amino acid sequence of SEQ ID NO: 11. Sequence was produced using the reverse translation tool located at: www.vivo.colostate.edu/molkit/rtranslate/index.html.

SEQ ID NO: 23 is the amino acid sequence of a positively charged EBD domain, EBD(+), which is a random sequence containing disorder-promoting residues P, Q, S and K in about the following amino acid ratios: K:P:Q:S=1:2:1:2. The sequence was produced using the random sequence generator tool located at the Swiss-Prot website: http://au.expasy.org/tools/randseq.html.

SEQ ID NO: 24 is the amino acid sequence of a positively charged EBD domain of SEQ ID NO: 23.

SEQ ID NO: 25 is the amino acid sequence of a negatively charged EBD domain, EBD(−), which is a random sequence containing disorder-promoting residues P, Q, S and E in about the following amino acid ratios: E:P:Q:S=1:2:1:2. The sequence was produced using the random sequence generator tool located at the Swiss-Prot website: http://au.expasy.org/tools/randseq.html.

SEQ ID NO: 26 is the amino acid sequence of a positively charged EBD domain of SEQ ID NO: 25.

SEQ ID NO: 27 is the amino acid sequence of a neutral EBD domain, EBD(0), which is a random sequence containing disorder-promoting residues P, Q, S and G in about the following amino acid ratios: G:P:Q:S=1:2:1:2. The sequence was produced using the random sequence generator tool located at the Swiss-Prot website: http://au.expasy.org/tools/randseq.html.

SEQ ID NO: 28 is the amino acid sequence of a positively charged EBD domain of SEQ ID NO: 27.

SEQ ID NO: 29 is a polynucleotide sequence encoding the amino acid sequence of SEQ ID NO: 23. Sequence was produced using the reverse translation tool located at: www.vivo.colostate.edu/molkit/rtranslate/index.html.

SEQ ID NO: 30 is a polynucleotide sequence encoding the amino acid sequence of SEQ ID NO: 24. Sequence was produced using the reverse translation tool located at: www.vivo.colostate.edu/molkit/rtranslate/index.html.

SEQ ID NO: 31 is a polynucleotide sequence encoding the amino acid sequence of SEQ ID NO: 25. Sequence was produced using the reverse translation tool located at: www.vivo.colostate.edu/molkit/rtranslate/index.html.

SEQ ID NO: 32 is a polynucleotide sequence encoding the amino acid sequence of SEQ ID NO: 26. Sequence was produced using the reverse translation tool located at: www.vivo.colostate.edu/molkit/rtranslate/index.html.

SEQ ID NO: 33 is a polynucleotide sequence encoding the amino acid sequence of SEQ ID NO: 27. Sequence was produced using the reverse translation tool located at: www.vivo.colostate.edu/molkit/rtranslate/index.html.

SEQ ID NO: 34 is a polynucleotide sequence encoding the amino acid sequence of SEQ ID NO: 28. Sequence was produced using the reverse translation tool located at: www.vivo.colostate.edu/molkit/rtranslate/index.html.

### DETAILED DESCRIPTION OF THE INVENTION

Artificial EBD fusion polynucleotides, polypeptides and vectors are provided by the present invention which offers significant advantages in the context of recombinant polypeptide production, particularly where it is desired to achieve, for example, improved solubility, improved yield, improved folding and/or reduced aggregation of a recombinant polypeptide of interest.

Artificial EBDs take advantage of the unique features of different classes of amino acids that are found within regions of order and disorder. The amino acids compositions of disordered and ordered regions in proteins are significantly different. Based on the analysis of intrinsically disordered proteins and regions within proteins, amino acids can be grouped into 3 categories: 1) order-promoting, 2) disorder-promoting, and 3) neutral (Dunker et al., Intrinsically disordered protein. J Mol Graph Model, 2001. 19(1): p. 26-59).

The advantages of the present invention are made possible by proper selection of disorder-promoting residues, order-promoting residues and/or neutral residues, as well as their respective proportions, within an artificial EBD sequence, as described herein. Proteins which have proven difficult to produce by conventional recombinant methodologies can be successfully produced when employing the artificial EBD sequences of the present invention.

The term “disorder-promoting amino acid residue” means an amino acid residue that promotes the disorder of stable tertiary and/or secondary structure within a polypeptide in solution. Disorder-promoting residues include D, M, K, R, S, Q, P, E and G.

The term “order-promoting amino acid residue” means an amino acid residue that promotes stable tertiary and/or secondary structure within a polypeptide in solution. Order-promoting amino acid residues include C, W, Y, I, F, V, L, H, T and N.

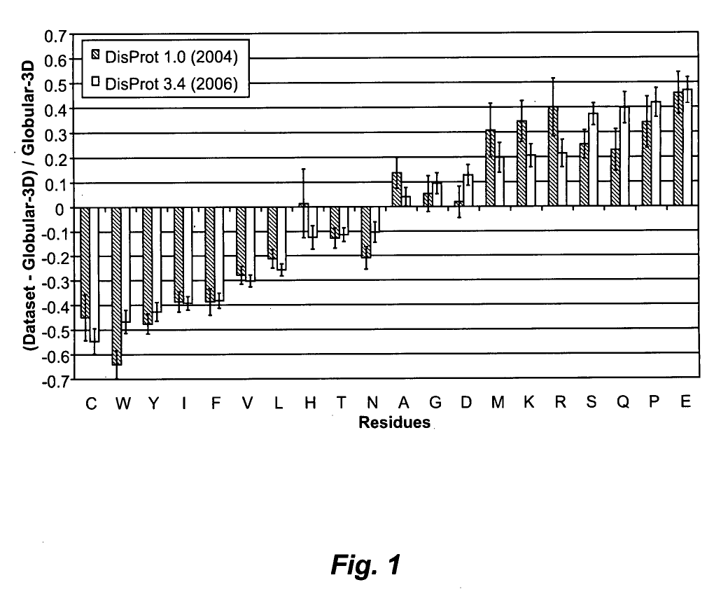

Neutral amino acid residues include A. The class of neutral amino acids can also include H, T, N, G, and D, as these amino acids tend to influence the tertiary and/or secondary structures within a protein or polypeptide to a relatively lesser extent then the other amino acids residues in above-defined classes (FIG. 1).

The phrases “about the ratio” and “in about the following amino acid ratio” means a group of amino acids as described herein, wherein the range “about” is determined by the actual ratio of said group of amino acids, first normalized by the lowest integer value within said group and then rounded to the nearest integer value. The resulting ratio if identical to the claimed ratio is then said to be “about” the claimed ratio of the group of amino acids. For example, consider a 100 AA EBD sequence of a fusion polypeptide which has the actual amino acid ratio of X:P:Q:S of 30:26:14:32. The actual amino acid ratio is normalized to 14, the lowest integer value, to yield a ratio of 2.1:1.9:1:2.3, which rounded to the nearest integer value is the ratio 2:2:1:2. Thus, a 100 AA EBD domain with an actual ratio of 30:26:14:32 has about the following amino acid ratio X:P:Q:S=2:2:1:2.

As used herein, the terms “polypeptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. Polypeptides of the invention may be prepared using any of a variety of well known recombinant and/or synthetic techniques, illustrative examples of which are further discussed below.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Fusion polypeptides comprising an EBD sequence and a heterologous polypeptide exhibit improved solubility relative to the corresponding heterologous polypeptide in the absence of the EBD sequence. In one embodiment, for example, the fusion polypeptide has at least 5% increased solubility relative to the heterologous polypeptide sequence alone. In another related embodiment, the fusion polypeptide has at least 25% increased solubility relative to the heterologous polypeptide sequence. In yet another related embodiment, the fusion polypeptide has at least 50% increased solubility relative to the heterologous polypeptide sequence.

The extent of improved solubility provided by an EBD sequence described herein can be determined using any of a number of available approaches (see for example, Kapust, R. B. and D. S. Waugh, Escherichia coli maltose-binding protein is uncommonly effective at promoting the solubility of polypeptides to which it is fused. Protein Sci, 1999. 8:1668-74; Fox, J. D., et al., Maltodextrin-binding proteins from diverse bacteria and archaea are potent solubility enhancers. FEBS Lett, 2003. 537:53-7; Dyson M R, Shadbolt S P, Vincent K J, Perera R L, McCafferty J. Production of soluble mammalian proteins in Escherichia coli: identification of protein features that correlate with successful expression. BMC Biotechnol. 2004 Dec. 14; 4(1):32).

Cells from single, drug resistant colony of E. coli overproducing the fusion polypeptide are grown to saturation in LB broth (Miller J H. 1972. Experiments in molecular genetics. Cold Spring Harbor, N.Y.: Cold Spring Harbor Press. p 433) supplemented with 100 mg/mL ampicillin and 30 mg/mL chloramphenicol at 37° C. The saturated cultures are diluted 50-fold in the same medium and grown in shake-flasks to mid-log phase (A₆₀₀ ˜0.5-0.7), at which time IPTG is added to a final concentration of 1 mM. After 3 h, the cells are recovered by centrifugation. The cell pellets are resuspended in 0.1 culture volumes of lysis buffer (50 mM Tris-HCl (pH 8.0), 150 mM NaCl, 1 mM EDTA), and disrupted by sonication. A total protein sample is collected from the cell suspension after sonication, and a soluble protein sample is collected from the supernatant after the insoluble debris is pelleted by centrifugation (20,000×g). These samples are subjected to SDS-PAGE and proteins are visualized by staining with Coomassie Brilliant Blue. At least three independent experiments are typically performed to obtain numerical estimates of the solubility of each fusion protein in E. coli. Coomassie-stained gels will be scanned with a gel-scanning densitometer and the pixel densities of the bands corresponding to the fusion proteins are obtained directly by volumetric integration. In each lane, the collective density of all E. coli proteins that are larger than the largest fusion protein are also determined by volumetric integration and used to normalize the values in each lane relative to the others. The percent solubility of each fusion protein is calculated by dividing the amount of soluble fusion protein by the total amount of fusion protein in the cells, after first subtracting the normalized background values obtained from negative control lanes (cells containing no expression vector). Descriptive statistical data (e.g., the mean and standard deviation) is then generated using standard methods.

The presence of an EBD sequence in fusion polypeptides of the present invention can also serve to reduce the extent of aggregation of a heterologous polypeptide sequence. In one embodiment, for example, the fusion polypeptide exhibits at least 10% reduced aggregation relative to the heterologous polypeptide. In another embodiment, the fusion polypeptide has at least 25% reduced aggregation relative to the heterologous polypeptide.

The extent of reduced aggregation provided by the fusion polypeptides of the present invention can be determined using any of a number of available techniques (see for example, Kapust, R. B. and D. S. Waugh, Escherichia coli maltose-binding protein is uncommonly effective at promoting the solubility of polypeptides to which it is fused. Protein Sci, 1999. 8:1668-74; Fox, J. D., et al., Maltodextrin-binding proteins from diverse bacteria and archaea are potent solubility enhancers. FEBS Lett, 2003. 537:53-7).

Cells from single, drug resistant colony of E. Coli overproducing the fusion polypeptide are grown to saturation in LB broth (Miller J H. 1972. Experiments in molecular genetics. Cold Spring Harbor, N.Y.: Cold Spring Harbor Press. p 433) supplemented with 100 mg/mL ampicillin and 30 mg/mL chloramphenicol at 37° C. The saturated cultures are diluted 50-fold in the same medium and grown in shake-flasks to mid-log phase (A₆₀₀ ˜0.5-0.7), at which time IPTG is added to a final concentration of 1 mM. After 3 h, the cells are recovered by centrifugation. The cell pellets are resuspended in 0.1 culture volumes of lysis buffer (50 mM Tris-HCl (pH 8.0), 150 mM NaCl, 1 mM EDTA), and disrupted by sonication. A total protein sample is collected from the cell suspension after sonication, and an insoluble protein sample is collected from the pellet after the centrifugation (20,000×g). These samples are subjected to SDS-PAGE and proteins are visualized by staining with Coomassie Brilliant Blue. At least three independent experiments are typically performed to obtain numerical estimates of the solubility of each fusion protein in E. coli. Coomassie-stained gels are scanned with a gel-scanning densitometer and the pixel densities of the bands corresponding to the fusion proteins are obtained directly by volumetric integration. In each lane, the collective density of all insoluble E. coli proteins that are larger than the largest fusion protein is also determined by volumetric integration and used to normalize the values in each lane relative to the others. The percent insolubility of each fusion protein is calculated by dividing the amount of insoluble fusion protein by the total amount of fusion protein in the cells, after first subtracting the normalized background values obtained from negative control lanes (cells containing no expression vector). Descriptive statistical data (e.g., the mean and standard deviation) is generated by standard methods.

The presence of an EBD sequence in the fusion polypeptides of the present invention can also serve to improve the folding characteristics of the fusion polypeptides relative to the corresponding heterologous polypeptide, e.g., by minimizing interference caused by interaction with other proteins.

Assays for evaluating the folding characteristics of a fusion polypeptide of the invention can be carried out using conventional techniques, such as circular dichroism spectroscopy in far ultra-violet region, circular dichroism in near ultra-violet region, nuclear magnetic resonance spectroscopy, infra-red spectroscopy, Raman spectroscopy, intrinsic fluorescence spectroscopy, extrinsic fluorescence spectroscopy, fluorescence resonance energy transfer, fluorescence anisotropy and polarization, steady-state fluorescence, time-domain fluorescence, numerous hydrodynamic techniques including gel-filtration, viscometry, small-angle X-ray scattering, small angle neutron scattering, dynamic light scattering, static light scattering, scanning microcalorimetry, and limited proteolysis.

In another embodiment of the invention, an EBD comprises an amino acid sequence that maintains a substantially random coil conformation. Whether a given amino acid sequence maintains a substantially random coil conformation can be determined by circular dichroism spectroscopy in far ultra-violet region, nuclear magnetic resonance spectroscopy, infra-red spectroscopy, Raman spectroscopy, fluorescence spectroscopy, numerous hydrodynamic techniques including gel-filtration, viscometry, small-angle X-ray scattering, small angle neutron scattering, dynamic light scattering, static light scattering, scanning microcalorimetry, and limited proteolysis.

In another embodiment of the invention, an EBD sequence comprises an amino acid sequence that is substantially mutually repulsive. This property of being mutually repulsive can be determined by simple calculations of charge distribution within the polypeptide sequence.

In yet another embodiment of the invention, an EBD sequence comprises an amino acid sequence that remains in substantially constant motion, particularly in an aqueous environment. The property of being in substantially constant motion can be determined by nuclear magnetic resonance spectroscopy, small-angle X-ray scattering, small angle neutron scattering, dynamic light scattering, intrinsic fluorescence spectroscopy, extrinsic fluorescence spectroscopy, fluorescence resonance energy transfer, fluorescence anisotropy and polarization, steady-state fluorescence, time-domain fluorescence.

In another embodiment, the fusion polypeptides of the invention further comprise independent cleavable linkers, which allow an EBD sequence, for example at either the N or C terminus, to be easily cleaved from a heterologous polypeptide sequence of interest. Such cleavable linkers are known and available in the art. This embodiment thus provides improved isolation and purification of a heterologous polypeptide sequence and facilitates downstream high-throughput processes.

The present invention also provides polypeptide fragments of an EBD polypeptide sequence described herein, wherein the fragment comprises at least about 5, 10, 15, 20, 25, 50, or 100 contiguous amino acids, or more, including all intermediate lengths, of an EBD polypeptide sequence set forth herein, or those encoded by a polynucleotide sequence set forth herein. In a preferred embodiment, an EBD fragment provides similar or improved activity relative to the activity of the EBD sequence from which it is derived (wherein the activity includes, for example, one or more of improved solubility, improved folding, reduced aggregation and/or improved yield, when in fusion with a heterologous polypeptide sequence of interest.

In another aspect, the present invention provides variants of an EBD polypeptide sequence described herein. EBD polypeptide variants will typically exhibit at least about 70%, 75%, 80%, 85%, 90%, 91%, 92%, 93%, 94%, 95%, 96%, 97%, 98%, or 99% or more identity (e.g., determined as described below), along its length, to an EBD polypeptide sequence set forth herein. Preferably the EBD variant provides similar or improved activity relative to the activity of the EBD sequence from which the variant was derived (wherein the activity includes one or more of improved solubility, improved folding, reduced aggregation and/or improved yield, when in fusion with a heterologous polypeptide sequence of interest.

An EBD polypeptide variant thus refers to a polypeptide that differs from an EBD polypeptide sequence disclosed herein in one or more substitutions, deletions, additions and/or insertions. Such variants may be naturally occurring or may be synthetically generated, for example, by modifying one or more of the EBD polypeptide sequences of the invention and evaluating their activity as described herein and/or using any of a number of techniques well known in the art.

In certain instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the EBD polynucleotides and polypeptides of the present invention and still obtain a functional molecule that encodes a variant or derivative polypeptide with desirable activity. When it is desired to alter the amino acid sequence of an EBD polypeptide to create an equivalent or an improved EBD variant or EBD fragment, one skilled in the art can readily change one or more of the codons of the encoding DNA sequence, for example according to Table 1.

For example, certain amino acids may be substituted for other amino acids in a protein structure without appreciable loss of desired activity. It is thus contemplated that various changes may be made in the EBD polypeptide sequences of the invention, or corresponding DNA sequences which encode said EBD polypeptide sequences, without appreciable loss of their desired activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |

| | | | | | | |
|---|---|---|---|---|---|---|
| Threonine | Thr | T | ACA | ACC | ACG | ACU |
| Valine | Val | V | GUA | GUC | GUG | GUU |

| Tryptophan | Trp | W | UGG | |
|---|---|---|---|---|
| Tyrosine | Tyr | Y | UAC | UAU |

In making such changes, the hydropathic index of amino acids may also be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn has potential bearing on the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Therefore, according to certain embodiments, amino acids within an EBD sequence of the invention may be substituted by other amino acids having a similar hydropathic index or score. Preferably, any such changes result in an EBD sequence with a similar level of activity as the unmodified EBD sequence. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Thus, an amino acid can be substituted for another having a similar hydrophilicity value and in many cases still retain a desired level of activity. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like.

Amino acid substitutions within an EBD sequence of the invention may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

In an illustrative embodiment, a variant EBD polypeptide differs from the corresponding unmodified EBD sequence by substitution, deletion or addition of five percent of the original amino acids or fewer. Variants may also (or alternatively) be modified by, for example, the deletion or addition of amino acids that have minimal influence on the desired activity.

A polypeptide of the invention may further comprise a signal (or leader) sequence at the N-terminal end of the polypeptide, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support.

As noted above, the present invention provides EBD polypeptide variant sequences which share some degree of sequence identity with an EBD polypeptide specifically described herein, such as those having at least 40%, 50%, 60%, 70%, 80%, 90% or 95% identity with an EBD polypeptide sequence described herein. When comparing polypeptide sequences to evaluate their extent of shared sequence identity, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O., (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes, pp. 626-645 Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Saitou, N. Nei, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

In another aspect of the invention, there is provided an isolated polynucleotide sequence encoding a fusion polypeptide, the fusion polypeptide comprising at least one EBD sequence and at least one heterologous polypeptide sequence of interest. In a related aspect, the invention provides expression vectors comprising a polynucleotide encoding an EBD fusion polypeptide of the invention. In another related aspect, an expression vector of the invention comprises a polynucleotide encoding one or more EBD sequence and further comprises a multiple cloning site for the insertion of a polynucleotide encoding a heterologous polypeptide sequence of interest.

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references).

In addition, any polynucleotide of the invention, such as a polynucleotide encoding an EBD polypeptide sequence, or a vector comprising a polynucleotide encoding an EBD polypeptide sequence, may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

The terms “DNA” and “polynucleotide” are used essentially interchangeably herein to refer to a DNA molecule that has been isolated free of total genomic DNA of a particular species. “Isolated”, as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will also be recognized, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

In addition to the EBD polynucleotide sequences set forth herein, the present invention also provides EBD polynucleotide variants having substantial identity to an EBD polynucleotide sequence disclosed herein, for example those comprising at least 50% sequence identity, preferably at least, 55%, 60%, 65%, 70%, 75%, 80%, 85%, 90%, 95%, 96%, 97%, 98%, or 99% or higher, sequence identity compared to an EBD polynucleotide sequence of this invention using the methods described herein, (e.g., BLAST analysis using standard parameters, as described below). One skilled in this art will recognize that these values can be appropriately adjusted to determine corresponding identity of polypeptides encoded by two polynucleotide sequences by taking into account codon degeneracy, amino acid similarity, reading frame positioning and the like.

Typically, EBD polynucleotide variants will contain one or more substitutions, additions, deletions and/or insertions, preferably such that the activity (e.g., improved folding, reduced aggregation and/or improved yield, when in fusion with a heterologous sequence of interest) of the polypeptide encoded by the variant polynucleotide is not substantially diminished relative to the corresponding unmodified polynucleotide sequence.

In additional embodiments, the present invention provides polynucleotide fragments comprising or consisting of various lengths of contiguous stretches of sequence identical to or complementary to one or more of the EBD polynucleotide sequences disclosed herein. For example, polynucleotides are provided by this invention that comprise or consist of at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence. Preferably, an EBD polynucleotide fragment of the invention encodes a fusion polypeptide that retains one or more desired activities, e.g., improved folding, reduced aggregation and/or improved yield, when in fusion with a heterologous sequence of interest.

The EBD polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

It will be appreciated by those of ordinary skill in the art that, as a result of the degeneracy of the genetic code, there are many nucleotide sequences that will encode a polypeptide as described herein. Some of these polynucleotides bear minimal homology to the native polynucleotide sequence. Nonetheless, polynucleotides that vary due to differences in codon usage are specifically contemplated by the present invention. Further, different alleles of an EBD polynucleotide sequence provided herein are within the scope of the present invention. Alleles are endogenous sequences that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

In another embodiment of the invention, a mutagenesis approach, such as site-specific mutagenesis, may be employed for the preparation of variants and/or derivatives of the EBD polynucleotides and polypeptides described herein. By this approach, for example, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the present invention contemplates the mutagenesis of the disclosed polynucleotide sequences to alter one or more activities/properties of the encoded polypeptide. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length may be employed, in about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention wherein one or more desired activities is improved or modified.

In other embodiments of the present invention, the polynucleotide sequences provided herein can be advantageously used as probes or primers for nucleic acid hybridization. As such, it is contemplated that nucleic acid segments that comprise or consist of a sequence region of at least about a 15 nucleotide long contiguous sequence that has the same sequence as, or is complementary to, a 15 nucleotide long contiguous sequence disclosed herein may be used. Longer contiguous identical or complementary sequences, e.g., those of about 20, 30, 40, 50, 100, 200, 500, 1000 (including all intermediate lengths) and even up to full length sequences will also be of use in certain embodiments.

Many template dependent processes are available to amplify a target sequence of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

As noted, the EBD fusion polynucleotides, polypeptides and vectors of the present invention are advantageous in the context of recombinant polypeptide production, particularly where it is desired to achieve, for example, improved solubility, improved yield, improved folding and/or reduced aggregation of a heterologous polypeptide to which an EBD polypeptide sequence has been operably fused. Therefore, another aspect of the invention provides methods for producing a recombinant protein, for example by introducing into a host cell an expression vector comprising a polynucleotide sequence encoding a fusion polypeptide as described herein, e.g., a fusion polypeptide comprising at least one EBD sequence and at least one heterologous polypeptide sequence of interest; and expressing the fusion polypeptide in the host cell. In a related embodiment, the method further comprises the step of isolating the fusion polypeptide from the host cell. In another embodiment, the method further comprises the step of removing an EBD sequence from the fusion polypeptide before or after isolating the fusion polypeptide from the host cell.

For recombinant production of a fusion polypeptide of the invention, DNA sequences encoding the polypeptide components of a fusion polypeptide (e.g., one or more EBD sequences and a heterologous polypeptide sequence of interest) may be assembled using conventional methodologies. In one example, the components may be assembled separately and ligated into an appropriate expression vector. For example, the 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the activities of both component polypeptides.

A peptide linker sequence may be employed to separate an EBD polypeptide sequence from a heterologous polypeptide sequence by some defined distance, for example a distance sufficient to ensure that the advantages of the invention are achieved, e.g., advantages such as improved folding, reduced aggregation and/or improved yield. Such a peptide linker sequence may be incorporated into the fusion polypeptide using standard techniques well known in the art. Suitable peptide linker sequences may be chosen based, for example, on the factors such as: (1) their ability to adopt a flexible extended conformation; and (2) their inability to adopt a secondary structure that could interfere with the activity of the EBD sequence. Illustrative peptide linker sequences, for example, may contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length, for example.

The ligated DNA sequences of a fusion polynucleotide are operably linked to suitable transcriptional and/or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The EBD and heterologous polynucleotide sequences may comprise a sequence as described herein, or may comprise a sequence that has been modified to facilitate recombinant polypeptide production. As will be understood by those of skill in the art, it may be advantageous in some instances to produce polypeptide-encoding polynucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In a particular embodiment, a fusion polynucleotide is engineered to further comprise a cleavage site located between the EBD polypeptide-encoding sequence and the heterologous polypeptide sequence, so that the hetereolous polypeptide may be cleaved and purified away from an EBD polypeptide sequence at any desired stage following expression of the fusion polypeptide. Illustratively, a fusion polynucleotide of the invention may be designed to include heparin, thrombin, or factor Xa protease cleavage sites.

In order to express a desired polypeptide, the nucleotide sequences encoding the polypeptide, or functional equivalents, may be inserted into appropriate expression vector, i.e., a vector which contains the necessary elements for the transcription and translation of an inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences of the present invention. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like. Proteins made in such systems may be designed to include heparin, thrombin, or factor Xa protease cleavage sites so that the cloned polypeptide of interest can be released from the EBD moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91:3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinothricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the polypeptide from cell culture. The polypeptide produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to polynucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor Xa or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. Further discussion of vectors which comprise fusion proteins can be found in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Polypeptide synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

According to another aspect, the present invention further provides binding agents, such as antibodies and antigen-binding fragments thereof, that specifically bind to an EBD sequence according to the present invention, or to a portion, variant or derivative thereof. Such binding agents may be used, for example, to detect the presence of a polypeptide comprising an EBD sequence, to facilitate purification of a polypeptide comprising an EBD sequence, and the like. An antibody, or antigen-binding fragment thereof, is said to “specifically bind” to a polypeptide if it reacts at a detectable level (within, for example, an ELISA assay) with the polypeptide, and does not react detectably with unrelated polypeptides under similar conditions.

Antibodies and other binding agents can be prepared using conventional methodologies. For example, monoclonal antibodies specific for a polypeptide of interest may be prepared using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

Yet another aspect of the invention provides kits comprising one or more compositions described herein, e.g., an isolated EBD polynucleotide, polypeptide, antibody, vector, host cell, etc. In a particular embodiment, the invention provides a kit containing an expression vector comprising a polynucleotide sequence encoding an EBD polypeptide sequence and a multiple cloning site for easily introducing into the vector a polynucleotide sequence encoding a heterologous polypeptide sequence of interest. In another embodiment, the expression vector further comprises an engineered cleavage site to facilitate separation of the EBD polypeptide sequence from the heterologous polypeptide sequence of interest following recombinant production.

The following Examples are offered by way of illustration and not by way of limitation.

### EXAMPLES

### Example 1

### Artificial EBDs Effectively Solubilize Insoluble Proteins

To address host cell toxicity problems associated with the use of certain naturally-occurring EBD sequences in fusion with heterologous proteins, artificial sequences were designed. Our knowledge of the intrinsic protein disorder phenomenon allowed us to design highly disordered artificial EBD sequences with desirable charge properties. Further, the likelihood that a completely artificial sequence would possess cytotoxicity due to the specific interaction with cellular components seemed to be minimal.

### Designing the Artificial Entropic Bristles

In order to serve as an artificial EBD, a polypeptide chain should be highly flexible and disordered. Statistical comparisons of amino acid compositions indicated that disordered and ordered regions in proteins are different to a significant degree. Based on the analysis of intrinsically disordered (ID) proteins and disordered regions within proteins, amino acid residues were categorized as (1) order-promoting, (2) disorder-promoting and (3) neutral (Dunker, et al., J Mol Graph Model, 2001. 19(1): p. 26-59). FIG. 1 presents relative amino acid compositions of ID regions available in the DisProt database (Sickmeier et al. Bioinformatics, 2005. 21(1): p. 137-40). The amino acid compositions were compared using a profiling approach (Dunker, et al., J Mol Graph Model, 2001. 19(1): p. 26-59). FIG. 1 shows that certain order-promoting residues include C, W, Y, I, F, V, L, H, T, and N, disorder-promoting residues include D, M, K, R, S, Q, P, E, and G, while neutral residues include A. It is notable that H, T, N, G, and D are borderline by the 0.1 fractional difference criterion, and so these residues could also be considered neutral in certain contexts.

The right-most bars representing the most disorder-promoting residues (E, P, Q, S, and K) together with the disorder-neutral residue G were chosen as basis for the de novo design of artificial EBDs. An artificial EBD was designed to contain the chosen residues in about the following amino acid ratios: X:P:Q:S=1:2:1:2, where X is a variable position to generate positive, negative or neutral bristles, and corresponds to one of K, E, or G, respectively.

The 1:2:1:2 proportions for X:P:Q:S were based on the following observations. Proline disrupts secondary structure (except for polyproline II helix) and contains hydrophobic surfaces for weak binding to possible aggregation patches, so a high proportion of P was chosen. PolyQ spontaneously aggregates, so a low proportion of Q was chosen to avoid aggregation-prone continuous stretches of Q. The side chain of serine is hydrophilic, but its ability to hydrogen bond with the backbone leads to very high conformational variability, so a high proportion of S was chosen. Since structured regions of proteins never contain long regions of very low complexity (Romero et al., Proteins. 2001. 42(1): p. 38-48), a small number of different amino acids (e.g., a low complexity bristle) reduces the chance of accidental formation of stable tertiary structure by stable interactions with other parts of the protein.

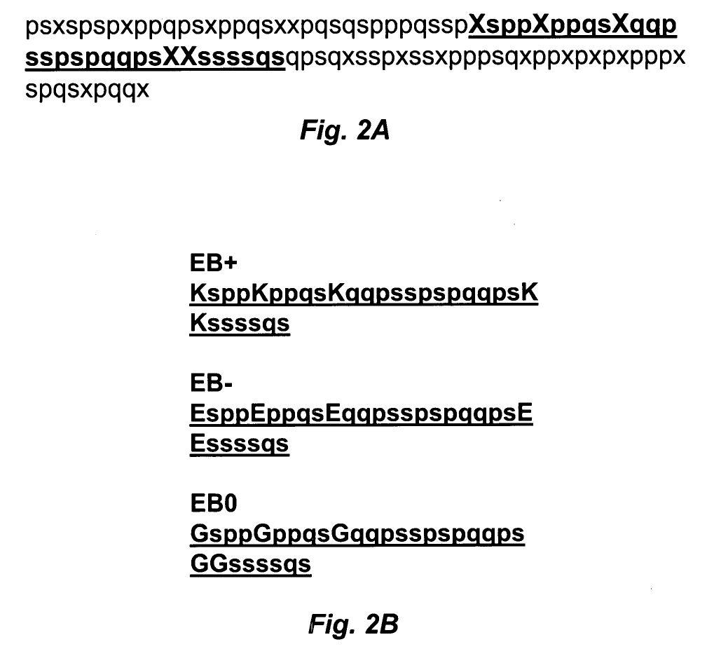

Based on these prerequisites, a 100 residue long random sequence was generated. The resulting sequence is shown in FIG. 2. Then, a fragment of this sequence, underlined sequence in FIG. 2A, was chosen to serve as the de novo EBD. This general sequence was used to generate EBDs that were positive (EB+), negative (EB−) and neutral (EB0) (FIG. 2B).

### Target Protein Selection

Thirteen proteins previously shown to be insoluble without fusions or shown to be insoluble even when fused to maltose-binding protein (MBP) were selected (Kapust et al., Protein Sci, 1999. 8(8): p. 1668-74; Kataeva et al., J Proteome Res, 2005. 4(6): p. 1942-51). Nine of these proteins were insoluble even at 30° C. of induction (Kataeva et al., J Proteome Res, 2005. 4(6): p. 1942-51). The proteins had molecular masses from 8.4 to 28.3 kDa; isoelectric points (pI) from 3.55 to 10.9, and net charges from +20 to −17. These proteins and some of their properties are listed in Table 2.

### Cloning Methods

To attach EBDs to N-termini of target proteins, the Gateway Cloning Technique (Invitrogen) based on a specific recombination of homologous DNA sequences was used. For polymerase chain reaction (PCR) accuracy, the high fidelity and specificity AccuPrime Pfx DNA polymerase (Invitrogen) was used (Takagi et al., Appl Environ Microbiol, 1997. 63(11): p. 4504-10). Primers were designed and optimized using XPression Primer 3.0 software. PCR products were purified using Wizard SV Gel and PCR Clean-Up System (Promega) or by mini-dialysis using Millipore. To generate entry clones, pDONR221 (Invitrogen) was used as an entry vector. All entry clones have been verified by sequencing. For the creation of expression clones, pDEST-42 destination vector (Gateway) was used. A point point mutation in pDEST-42 was done using QuickChange II XL Site-Directed Mutagenesis Kit (Stratagene). One Shot TOP10 and BL21 Star (DE3) One Shot competent cells (Invitrogen) were commonly used for transformation with BP and LR reactions, respectively. Plasmid DNAs were purified using Wizard Plus SV Minipreps DNA Purufication System (Promega). To create maltose-binding protein (MBP) fusions the target genes were amplified by PCR using forward and reverse primers flanked by attB1 and attB2 sites, respectively, and cloned into entry vector as described above. To create expression clones, pDEST-544 vector (Invitrogen) was used. Proteins expressed from this vector had an MBP at their N-termini.

### Cell Growth and Lysis

Cultures were grown in an LB medium supplied with 100 μg/mL ampicillin at 37° C. overnight and used next morning to start new 1 ml cultures. The tubes were incubated with shaking at 37° C. for 4 hours. Then IPTG was added to a final concentration of 1 mM and the tubes were shaken for additional 4 h at either 37° C. or 30° C. The cells were collected by centrifugation and lysed chemically using the combination of mild nonionic detergent and a lysozyme (B-PER Reagent, Pierce). The suspensions were stirred for 30 min at room temperature. The lysed solution was designated as a “whole fraction”. The “soluble fraction” was obtained by removal of insoluble fraction by centrifugation. The whole and the soluble fractions were used for the detection of protein expression and solubility, respectively.

### Design of Cloning Strategy

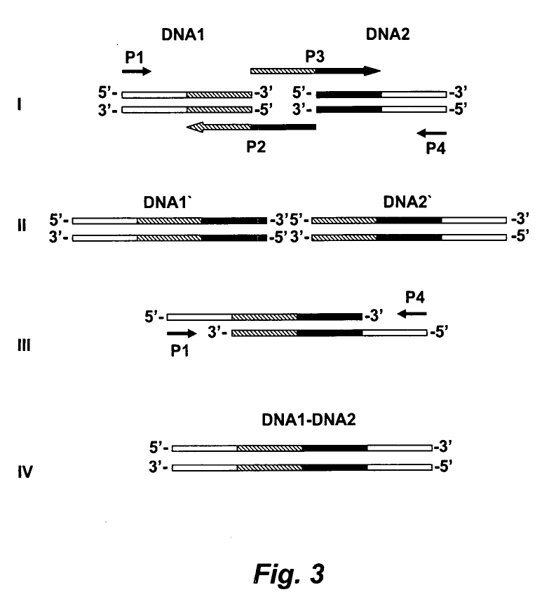

To avoid translation of the eleven amino acid residues attB1 recombination site, (i.e. for native protein expression), its start codon (ATG) was mutated to ATA encoding isoleucine. For the same reason, Shine-Dalgarno (SD) sequence followed by a linker (L) and a start codon were inserted between the attB1 site and the entropic bristle sequence. Original reversed transcripts of 30 amino acid residues of the designed artificial EBDs were 90 bases long. After addition of a 5′-fragment (the attB1 site, the Shine Dalgarno, the linker, and the start codon), the resulted DNA fragment to be synthesized was over 140 bases long. To minimize mistakes upon synthesis of such a large DNA fragment, the putative DNA sequence of each EBD was divided into three pieces. Each piece was amplified and linked to the next one using set of PCRs and overlapping primers (see FIG. 3) (Kataeva et al., J Proteome Res, 2005. 4(6): p. 1942-51). After generating of EBD DNA fragments, target genes with a stop codons at their 3′-termini were amplified by PCR and linked to the 3′-terminus of each entropic bristle using the above principle (FIG. 3). Thus, each final PCR product had the following composition: attB1-SD-L-EBD-Target Gene-stop-attB2. The constructs were inserted into cloning vector. Plasmid DNAs of the clones were isolated and verified by sequencing. The “right” clones were used (1) as sources of DNA sequences encoding EBDs and (2) to make expression clones in LR reaction.

### Expression and Solubility Test

To evaluate protein expression and solubility, the proteins of the whole and soluble fractions were separated by SDS-PAGE using NuPAGE 4-12% Bis-Tris Gels and the supplied reagents (Invitrogen). Gels were stained with Coomassie Blue Reagent.

### Results: Expression and Solubility of Fusion Proteins Comprising Artificial EBDs

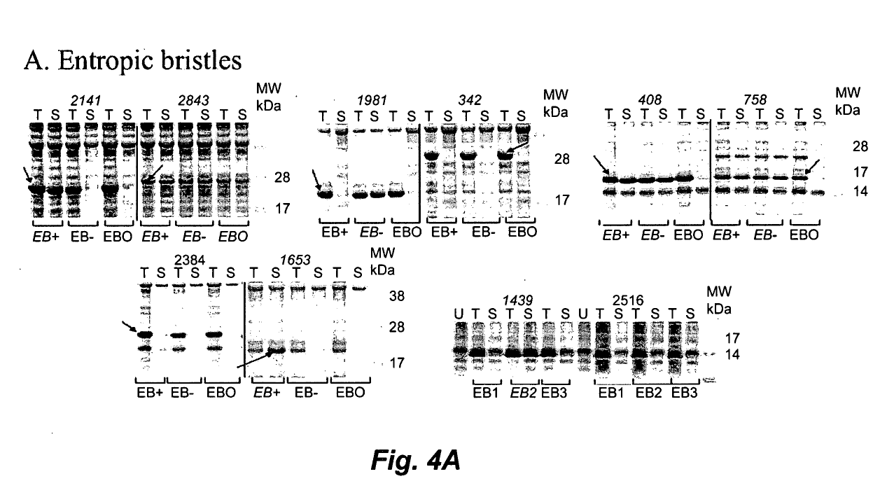

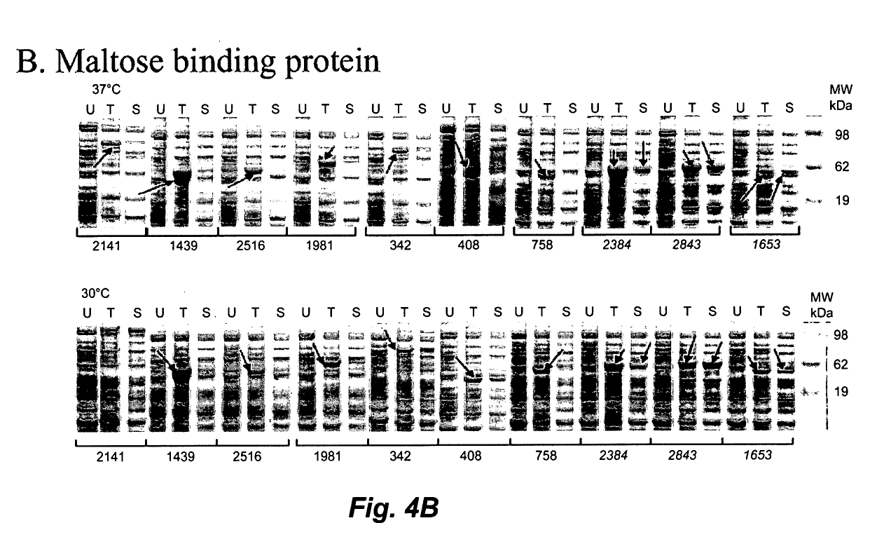

FIG. 4 and Table 2 show that artificial EBDs fused to the N-termini of target proteins was highly effective. Eleven out of thirteen insoluble proteins were solubilized by this approach (Highlighted portions of Table 2 represent the proteins that were solubilized by fusion to artificial EBDs or to MBP). The level of expression of all EBD-fusions was good. At 37° C. of induction, neutral EB0 solubilized 1 protein. Charged EB+ and EB− solubilized 5 and 6 proteins, respectively. Decreasing induction temperature improved soluble protein expression (Kataeva et al., J Proteome Res, 2005. 4(6): p. 1942-51). Induction at 30° C. did not change solubility of EBD0 fusions but resulted in 4 and 1 more soluble EBD+ and EBD− fusion proteins, respectively. FIG. 4 illustrates expression and solubility of 10 bacterial proteins fused either to artificial EBDs (FIG. 4A) or to maltose-binding protein (FIG. 4B), whereas Table 2 summarizes the results of the solubility studies.

TABLE 2

| | | | | 37° C. | | | | | | 30° C. | | | | | | 37° C. | | 30° C. | |
|---|---|---|---|---|---|---|---|---|---|---|---|---|---|---|---|---|---|---|---|
| | MW | | | EBD₊ | | EBD₋ | | EBD₀ | | EBD₊ | | EBD₋ | | EBD₀ | | MBP fusion | | | |
| Protein | (kDa) | pI | Charge | E | S | E | S | E | S | E | S | E | S | E | S | E | S | E | S |
| 342-Transposase_mut | 23.3 | 10.9 | 20 | 1 | 0 | 1 | 0 | 1 | 0 | 1 | 0 | 1 | 0 | 1 | 0 | 1 | 0 | 1 | 0 |
| 1981-IF-2B | 17.1 | 10 | 6.5 | 1 | 0 | 1 | 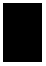 | 1 | 0 | 1 | 0 | 1 | 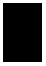 | 1 | 0 | 1 | 0 | 1 | 0 |
| 2516-DIF199 | 9.2 | 9.55 | 3 | 1 | 0 | 1 | 0 | 1 | 0 | 1 | 0 | 1 | 0 | 1 | 0 | 1 | 0 | 1 | 0 |
| 758-DUF111 | 12.5 | 7.3 | 5 | 1 | 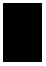 | 1 | 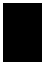 | 1 | 0 | 1 | 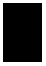 | 1 | 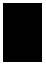 | 1 | 0 | 1 | 0 | 1 | 0 |
| 2843-Cons_hypoth95 | 21.7 | 6.8 | 0.5 | 1 | 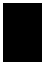 | 1 | 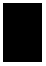 | 1 | 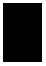 | 1 | 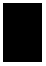 | 1 | 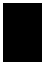 | 1 | 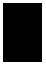 | 1 | 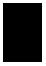 | 1 | 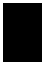 |
| 408-UbiA | 12.4 | 5.8 | −0.5 | 1 | 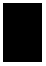 | 1 | 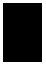 | 1 | 0 | 1 | 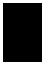 | 1 | 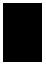 | 1 | 0 | 1 | 0 | 1 | 0 |
| 2384-HD | 21.1 | 5.5 | −1.5 | 1 | 0 | 1 | 0 | 1 | 0 | 1 | 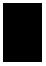 | 1 | 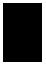 | 1 | 0 | 1 | 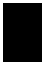 | 1 | 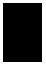 |
| CATΔ9 | 26.7 | 5.2 | −14 | 1 | 0 | 1 | 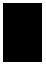 | 0 | 0 | 1 | 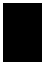 | 1 | 0 | 0 | 0 | 1 | 0 | 1 | 0 |
| 2141-DNA_gyraseB_C | 23.2 | 5.2 | −3 | 1 | 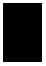 | 1 | 0 | 1 | 0 | 1 | 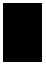 | 1 | 0 | 1 | 0 | 1 | 0 | 0 | 0 |
| GFP | 28.3 | 5.13 | −14 | 1 | 0 | 1 | 0 | 1 | 0 | 1 | 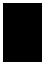 | 1 | 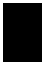 | 0 | 0 | 1 | 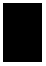 | 1 | 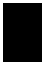 |
| p16 | 17.7 | 4.94 | −5 | 1 | 0 | 1 | 0 | 1 | 0 | 1 | 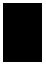 | 1 | 0 | 0 | 0 | 1 | 0 | 1 | 0 |
| 1653-UPF0004 | 17.1 | 4.4 | −8.5 | 1 | 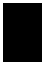 | 1 | 0 | 1 | 0 | 1 | 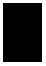 | 1 | 0 | 1 | 0 | 1 | 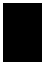 | 1 | 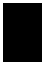 |
| 1439-AAA_div | 8.4 | 3.55 | −17 | 1 | 0 | 1 | 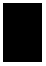 | 1 | 0 | 1 | 0 | 1 | 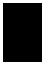 | 1 | 0 | 1 | 0 | 1 | 0 |

E = expression;
S = solubilization;
1 = soluble;
0 = insoluble

In summary, fusion of MBP significantly increased the solubility of just 4 of 13 proteins, at 37° C. or at 30° C., whereas the artificial EBD of the present invention increased the solubility for 11 of the 13 previously insoluble proteins.

What is claimed:

1. A fusion polypeptide comprising at least one non-naturally occurring entropic bristle domain (EBD) polypeptide sequence and at least one heterologous polypeptide sequence to be expressed, wherein the EBD polypeptide sequence is about 10-500 amino acid residues in length, and wherein at least 75% of the residues of the EBD polypeptide sequence are selected from G, D, M, K, R, S, Q, P, and E.

2. The fusion polypeptide of claim 1, wherein the fusion polypeptide has increased solubility relative to the heterologous polypeptide sequence, reduced aggregation relative to the heterologous polypeptide sequence and/or improved folding relative to the heterologous polypeptide sequence.

3. The fusion polypeptide of claim 1, wherein the EDB polypeptide sequence is about 25-300 amino acids in length.

4. The fusion polypeptide of claim 1, wherein the EDB polypeptide sequence is about 25-200 amino acids in length.

5. The fusion polypeptide of claim 1, wherein the EBD polypeptide sequence is positively charged and the amino acid residues are disorder-promoting amino acid residues selected from P, Q, S and K.

6. The fusion polypeptide of claim 5, wherein the disorder-promoting amino acid residues P, Q, S and K are present in about the following amino acid ratios: K:P:Q:S=1:2:1:2, K:P:Q:S=2:2:1:2, K:P:Q:S=3:2:1:2, K:P:Q:S=4:2:1:2, or K:P:Q:S=5:2:1:2.

7. The fusion polypeptide of claim 5, wherein the EDB polypeptide sequence comprises a sequence set forth in SEQ ID NO: 1, SEQ ID NO: 2, SEQ ID NO: 3, SEQ ID NO: 4, SEQ ID NO: 5, SEQ ID NO: 23, or SEQ ID NO: 24, or a fragment thereof, or a sequence having at least 90% identity thereto.

8. The fusion polypeptide of claim 1, wherein the EBD polypeptide sequence is negatively charged and the amino acid residues are disorder-promoting amino acid residues selected from P, Q, S and E.

9. The fusion polypeptide of claim 8, wherein the disorder-promoting amino acid residues P, Q, S and K are present in about the following amino acid ratios: E:P:Q:S=1:2:1:2, E:P:Q:S=2:2:1:2, E:P:Q:S=3:2:1:2, E:P:Q:S=4:2:1:2, or E:P:Q:S=5:2:1:2.

10. The fusion polypeptide of claim 8, wherein said EDB polypeptide comprises the sequence set forth in SEQ ID NO: 6, SEQ ID NO: 7, SEQ ID NO: 8, SEQ ID NO: 9, SEQ ID NO: 10, SEQ ID NO: 25, or SEQ ID NO: 26, or a fragment thereof, or a sequence having at least 90% identity thereto.

11. The fusion polypeptide of claim 1, wherein said EBD polypeptide sequence is neutral and the amino acid residues are selected from P, Q, S and G.

12. The fusion polypeptide of claim 11, wherein the disorder-promoting residues P, Q, S and G are present in about the amino acid ratio of G:P:Q:S=1:2:1:2.

13. The fusion polypeptide of claim 11, wherein said EDB polypeptide comprises the sequence set forth in SEQ ID NO: 11, SEQ ID NO: 27, or SEQ ID NO: 28, or a fragment thereof, or a sequence having at least 90% identity thereto.

14. The fusion polypeptide of claim 1, wherein said EBD polypeptide sequence is positively charged and the amino acid residues are disorder-promoting amino acid residues are selected from P, Q, S and R.

15. The fusion polypeptide of claim 14, wherein the amino acid residues R, P, Q and S are present in about the following amino acid ratios: R:P:Q:S=1:2:1:2, R:P:Q:S=2:2:1:2, R:P:Q:S=3:2:1:2, R:P:Q:S=4:2:1:2, or R:P:Q:S=5:2:1:2.

16. The fusion polypeptide of claim 1, wherein the EBD polypeptide sequence is negatively charged and the amino acid residues are disorder-promoting amino acid residues are selected from P, Q, S and D.

17. The fusion polypeptide of claim 16, wherein the amino acid residues D, P, Q and S are present in about the following amino acid ratios: D:P:Q:S=1:2:1:2, D:P:Q:S=2:2:1:2, D:P:Q:S=3:2:1:2, D:P:Q:S=4:2:1:2, or D:P:Q:S=5:2:1:2.

18. The fusion polypeptide of claim 1, wherein the fusion polypeptide further comprises a cleavable linker.

19. A polynucleotide encoding an EBD polypeptide sequence of claim 1.

20. A polynucleotide encoding a fusion polypeptide according to claim 1.

21. An expression vector comprising an isolated polynucleotide according to any one of claims 19 and 20.

22. A host cell comprising an expression vector according to claim 21.

23. A kit comprising a polynucleotide according to any one of claims 19 and 20, or a host cell according to claim 22.

24. A kit comprising an expression vector according to claim 21.

25. A method for producing a recombinant protein comprising the steps of: (a) introducing into a host cell a polynucleotide according to claim 20 or an expression vector according to claim 21; and (b) expressing in the host cell a fusion polypeptide comprising at least one EBD sequence and at least one heterologous polypeptide sequence.
